(19) United States
(12) Patent Application Publication
(10) Pub. No.: US 2026/0209174 A1
(43) Pub. Date: Jul. 23, 2026

(54) PROCESS FOR THE PREPARATION OF SULFONYLUREA BASED COMPOUNDS

(71) Applicant: Zydus Lifesciences Limited, Ahmedabad (IN)

(72) Inventors: Nirmal Kumar, Ahmedabad (IN); Rajiv Sharma, Ahmedabad (IN); Mukul R. Jain, Ahmedabad (IN)

(21) Appl. No.: 19/078,506

(22) Filed: Mar. 13, 2025

Related U.S. Application Data
(63) Continuation of application No. PCT/IB2023/059133, filed on Sep. 14, 2023.

(30) Foreign Application Priority Data
Sep. 14, 2022 (IN) 202221052473

(51) Int. Cl. C07D 207/08; C07C 209/68; C07C 211/61; C07C 273/18; C07C 275/28

(52) U.S. Cl. CPC C07D 207/08; C07C 209/68; C07C 211/61; C07C 273/1854; C07C 275/28

(57) ABSTRACT

The present invention relates to an improved process for the preparation of sulfonylurea based compounds of general formula (I). More specifically present invention provides an improved process for the preparation of compound of formula (I-a). Present invention also provides some novel impurities generated during process.

### CROSS-REFERENCE TO RELATED APPLICATIONS

This application is a continuation of International Application No. PCT/IB2023/059133, with an international filing date of Sep. 14, 2023, which claims the benefit of and priority to Indian Patent Application No. 202221052473, filed on Sep. 14, 2022, each of which is incorporated by reference herein in its entirety.

### FIELD OF THE INVENTION

The present invention relates to an improved process for the preparation of sulfonylurea based compounds of general formula (I). More specifically present invention provides an improved process for the preparation of compound of formula (I-a). Present invention also provides some novel impurities generated during process.

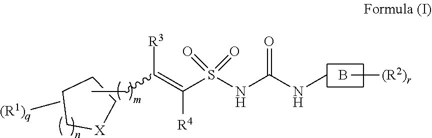

### BACKGROUND OF THE INVENTION

WIPO patent application no. WO2020148619 describes the use of certain sulfonylurea based compounds of formula (I) as NLRP3 modulators which are useful in the treatment of the diseases or conditions mediated by NLRP3 or conditions in which interleukin 13 activity is implicates. This includes inflammation, cryopyrin-associated periodic syndrome (CAPS), gouty arthritis, multiple sclerosis, inflammatory bowel diseases, Parkinson's and Alzheimer's diseases and other diseases related to Central Nervous System. NLRP3 modulators preferably useful as therapeutics in treatment of a variety of pathological condition including but not limited to lymphoma, auto-immune diseases, cancer, inflammatory diseases, neurodegenerative diseases or conditions. Compound of formula (I) was prepared according to scheme 1 & 2:

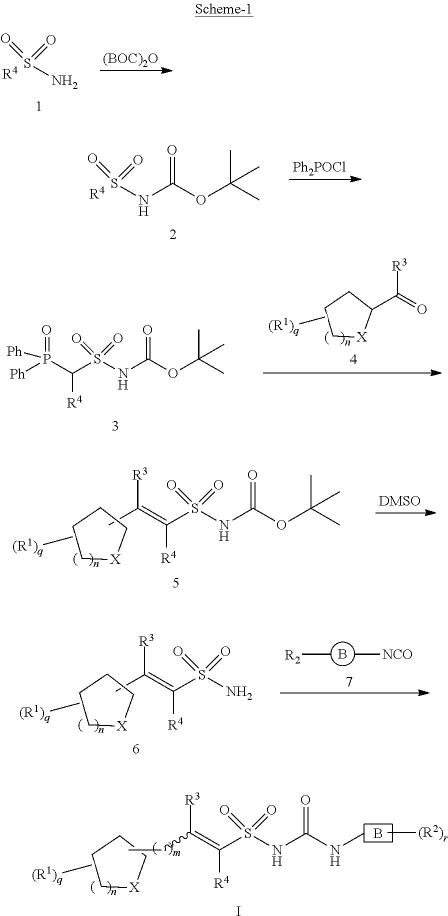

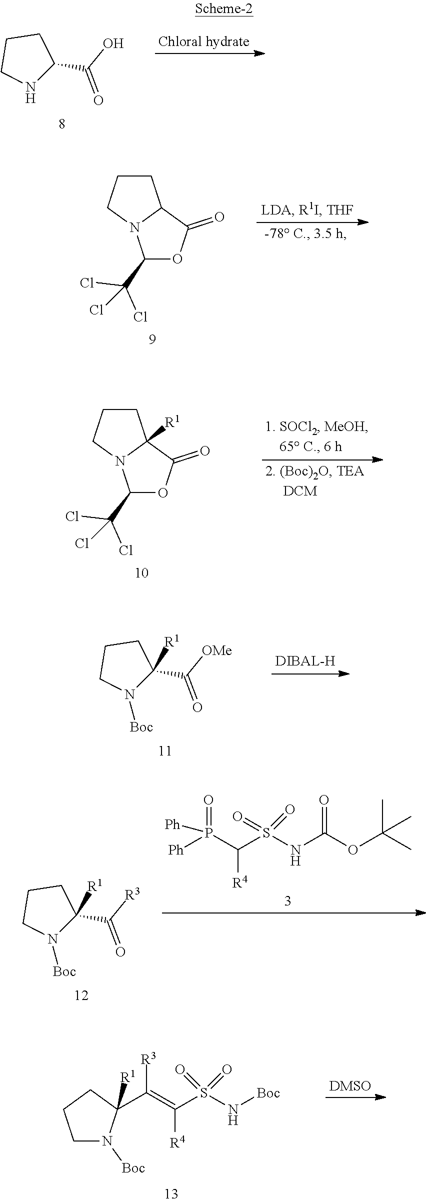

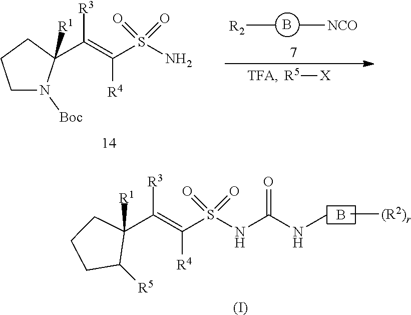

The drawback of process disclosed in WO2020148619 is that it teaches usage of many hazardous reagents and process requires column chromatographic purification using highly flammable solvent at one of the stage and purification at multi steps during synthesis, which is not feasible for bulk production.

Scheme 2 disclosed in WO2020148619 has Seebach approach to proceed with high selectivity, however, this required the use of unnatural D-proline as a very expensive starting material and cryogenic conditions with hazardous reagents such as n-Butyl lithium and diisobutylaluminium hydride. In addition, the reported procedure describes the formation of the acetal as challenging, requiring azeotropic distillation from pentane and very low yielding stages with low purity of final product. Therefore, there is need for an improved process for the preparation of (R, E)-2-(1,2-dimethylpyrrolidin-2-yl)-N-((1,2,3,5,6,7-hexahydro-s-indacen-4-yl)carbamoyl)ethene-1-sulfonamide formula (I-a) and intermediates thereof.

### SUMMARY OF THE INVENTION

The present invention discloses an improved process for the preparation of sulfonylurea based compounds of general formula (I). More specifically invention provides an improved process for the preparation of compound of formula (I-a). Present invention also provides some novel impurities generated during process. The compounds prepared by this method are useful for the diseases or conditions mediated by NLRP3 or conditions in which interleukin 13 activity is implicates.

### EMBODIMENTS OF THE INVENTION

In an embodiment, the present invention provides a process for the preparation of compound of formula (I)

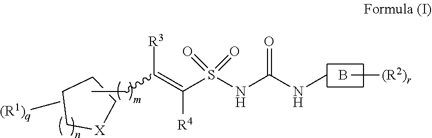

In another embodiment, the present invention provides a process for the preparation of compound of following formula (I-a).

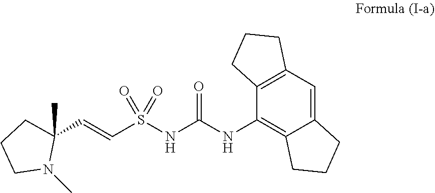 [SMILES: CN1CCC[C@]1(C)/C=C/S(=O)(=O)NC(=O)Nc1c2c(cc3c1CCC3)CCC2]

In an embodiment, the present invention provides a crystalline form of compound of formula (I-a).

In another embodiment, the present invention provides a process for the preparation of crystalline form of compound of formula (I-a).

In an embodiment is provided an amorphous form of compound of formula (I-a).

In another embodiment the present invention provides a process for the preparation of amorphous form of compound of formula (I-a).

In another embodiment, the present invention encompasses compounds having chemical name (R,E)-2-(1,2-dimethylpyrrolidin-2-yl)-N-((1,2,3,7-tetrahydro-s-indacen-4-yl)carbamoyl)ethene-1-sulfonamide (Compound A), (R)-2-(1,2-dimethylpyrrolidin-2-yl)-N-((1,2,3,5,6,7-hexahydro-s-indacen-4-yl)carbamoyl)ethane-1-sulfonamide (Compound B), chemical name 3-(1,2,3,5,6,7-hexahydro-s-indacen-4-yl)-1,1-dimethylurea (Compound C), chemical name 1,3-bis(1,2,3,5,6,7-hexahydro-s-indacen-4-yl)urea (Compound D), chemical name (S)-2-((R)-1,2-dimethylpyrrolidin-2-yl)-N-((1,2,3,5,6,7-hexahydro-s-indacen-4-yl)carbomoyl)-2-hydroxyethane-1-sulfonamide (Compound E), chemical name (R)-2-((R)-1,2-dimethylpyrrolidin-2-yl)-N-((1,2,3,5,6,7-hexahydro-s-indacen-4-yl)carbamoyl)-2-hydroxyethane-1-sulfonamide (Compound F), chemical name (R,E)-N-((1,2,3,5,6,7-hexahydro-s-indacen-4-yl)carbamoyl)-2-(2-methylpyrrolidin-2-yl)ethene-1-sulfonamide 2,2,2-trifluoroacetate (Compound G), chemical name 1,2,3,5,6,7-hexahydro-s-indacen-4-amine (Compound H), chemical name (R,E)-2-(1,2-dimethylpyrrolidin-2-yl)ethene-1-sulfonamide (Compound I), chemical name (S,E)-2-(1,2-dimethylpyrrolidin-2-yl)-N-((1,2,3,5,6,7-hexahydro-s-indacen-4-yl)carbamoyl)ethene-1-sulfonamide (Compound J) These compounds were formed as impurities during the process of preparing the compound of formula (I-a).

In another embodiment the compound of formula (A) is controlled in formula (I-a) with the limit of 0.01% to 2.0%.

In an embodiment the compound of formula (B) is controlled in formula (I-a) with the limit of 0.01% to 2.0%.

In an embodiment the compound of formula (C) is controlled in formula (I-a) with the limit of 0.01% to 2.0%.

In another embodiment the compound of formula (D) is controlled in formula (I-a) with the limit of 0.01% to 2.0%.

In another embodiment the compound of formula (E) is controlled in formula (I-a) with the limit of 0.01% to 2.0%.

In another embodiment the compound of formula (F) is controlled in formula (I-a) with the limit of 0.01% to 2.0%.

In another embodiment the compound of formula (G) is controlled in formula (I-a) with the limit of 0.01% to 2.0%.

In another embodiment the compound of formula (H) is controlled in formula (I-a) with the limit of 0.01% to 2.0%.

In another embodiment the compound of formula (I) is controlled in formula (I-a) with the limit of 0.01% to 2.0%.

In another embodiment the compound of formula (J) is controlled in formula (I-a) with the limit of 0.01% to 2.0%.

### BRIEF DESCRIPTION OF DRAWING

FIG. 1 is the Powder X-ray Diffraction pattern of crystalline Form I of (R,E)-2-(1,2-dimethylpyrrolidin-2-yl)-N-((1,2,3,5,6,7-hexahydro-s-indacen-4-yl)carbamoyl)ethene-1-sulfonamide (compound I-a).

FIG. 2 is the Powder X-ray Diffraction pattern of crystalline Form II of (R,E)-2-(1,2-dimethylpyrrolidin-2-yl)-N-((1,2,3,5,6,7-hexahydro-s-indacen-4-yl)carbamoyl)ethene-1-sulfonamide (compound I-a).

FIG. 3 is the Powder X-ray Diffraction pattern of crystalline Form III of (R,E)-2-(1,2-dimethylpyrrolidin-2-yl)-N-((1,2,3,5,6,7-hexahydro-s-indacen-4-yl)carbamoyl)ethene-1-sulfonamide (compound I-a).

FIG. 4 is the Powder X-ray Diffraction pattern of amorphous form (R,E)-2-(1,2-dimethylpyrrolidin-2-yl)-N-((1,2,3,5,6,7-hexahydro-s-indacen-4-yl)carbamoyl)ethene-1-sulfonamide (compound I-a).

### DETAILED DESCRIPTION OF THE INVENTION

Following is the list of abbreviations used in description:
CDCl₃: Deuterated chloroform
DCM: Dichloromethane
DBU: 1,8-Diazabicyclo[5,4,0]undec-7-ene
DBN: 1,5-Diazabicyclo[4.3.0]non-5-ene
DABCO: (1,4-diazabicyclo[2.2.2]octane)
DMF: Dimethylformamide
DMSO: Dimethyl sulfoxide
GC: Gas Chromatography
min: Minute(s)
MS: Mass Spectrometry
NMR: Nuclear Magnetic Resonance
HPLC: High Performance Liquid Chromatography
h: Hour
Kg: Kilogram
L: Liter
MeOD: Deuterated methanol
MS: Mass Spectrometry
MTBE: Methyl tert-butyl ether
NMR: Nuclear Magnetic Resonance
RT: Room Temperature [25-35° C.]
SOR: Specific Rotation
SSR: Stainless Steel Reactor
ESI-MS: Electrospray Ionization Mass Spectrometry

### Instrument Details:

HPLC and Chiral HPLC chromatograms were recorded on Agilent 1260 series.

Mass spectra were recorded on Waters XevoG2a-ToF LCMS instrument.

NMR was recorded with Bruker 400 MHZ instrument.

Specific rotation of chiral compounds was recorded with Jasco Polarimeter, Digital Polarimeter P-2000.

Spray drying was done on LAB SPRAY DRYER, LU 222 ADVANCED, SD-1000 The ‘aryl’ group used either alone or in combination with other radicals, is selected from a suitable aromatic system containing one two or three rings wherein such ring may be attached together in pendant manner or may be fused, more preferably the groups are selected from optionally substituted phenyl, naphthyl, tetrahydronaphthyl, biphenyl and the like;
the ‘alkyl’ group either used alone or in combination with other radicals, denotes a linear or branched radical containing one to six carbons, selected from methyl, ethyl, n-propyl, iso-propyl, n-butyl, sec-butyl, tert-butyl, amyl, t-amyl, n-pentyl, n-hexyl, and the like; the ‘alkoxy’ refers to the straight or branched chain alkoxides of the number of carbon atoms specified.
the ‘cycloalkyl’ group used either alone or in combination with other radicals, is selected from a cyclic radical containing three to six carbons, more preferably cyclopropyl, cyclobutyl, cyclopentyl, cyclohexyl, and the like;
the term “haloalkyl” means an alkyl structure in which at least one hydrogen is replaced with a halogen atom. In certain embodiments in which two or more hydrogen atoms are replaced with halogen atoms, the halogen atoms are all the same as one another.
the “haloalkoxy” group is selected from suitable haloalkyl, as defined above, directly attached to an oxygen atom, more preferably groups selected from fluoromethoxy, chloromethoxy, fluoroethoxy, chloroethoxy and the like;
the ‘heteroaryl’ means an aromatic or partially aromatic heterocycle that contains at least one ring heteroatom selected from O, S and N. Heteroaryls thus include heteroaryls fused to other kinds of ring's, such as aryls, cycloalkyls, and heterocycles that are not aromatic.

In certain other embodiment in which two or more hydrogen atoms are replaced with halogen atoms, the halogen atoms are not all the same as one another;

The ‘heterocyclyl’ means a saturated, partially saturated or unsaturated aromatic or non-aromatic mono, bi or tricyclic radicals, containing one or more heteroatoms selected from nitrogen, sulfur and oxygen, further optionally including the oxidized forms of sulfur, namely SO & SO₂ Heterocyclyl systems may be attached to another moiety via any number of carbon atoms or heteroatoms of the radical and may be both saturated and unsaturated.

The above and other objects of the present invention are attained as described herein below. In an embodiment, the objects are attained by the improved process for the preparation of sulfonylurea based compounds of the formula (I) and intermediates thereof as described herein.

In embodiment, there is provided a process for the preparation of compound of formula (I):

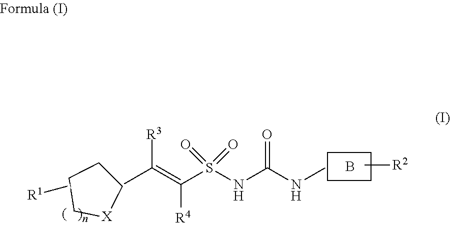

wherein
B described above may be selected from following ring system:

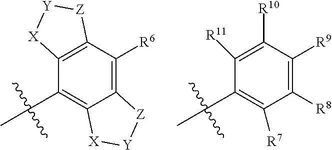

where in X, Y, Z at each occurrence independently represents C, N, S, SO₂, and O, which may be optionally substituted;
R¹ at each occurrence independently represents hydrogen, halogen, haloalkyl optionally substituted groups selected from (C₁-C₆)alkyl;
R² at each occurrence independently represents hydrogen, halogen, haloalkyl, optionally substituted groups selected from (C₁-C₆)alkyl;
R³ and R⁴ at each occurrence independently represents hydrogen;
X is N—R⁵; O, S, SO₂;
R⁵ at each occurrence independently represents hydrogen, halogen, haloalkyl, cyano, optionally substituted groups selected from (C₁-C₆)alkyl, (C₁-C₆)haloalkyl, (C₂-C₆)alkenyl, (C₂-C₆)alkynyl, (C₁-C₆)alkoxy, (C₃-C₇)cycloalkyl, (C₁-C₆)alkylSO₂(C₁-C₆)alkyl, (C₁-C₆)alkylN(C₁-C₆)alkyl, (C₁-C₆)alkylN(C₃-C₇)cycloalkyl, aryl, heteroaryl, heterocyclyl, benzyl, thiol, mercaptoalkyl, SO₂(C₁-C₆)alkyl, SO₂(C₃-C₇)cycloalkyl, SO₂-aryl, SO₂-heterocyclyl, (C₁-C₆)thioalkyl, (C₁-C₆)thioalkoxy, (C₁-C₆)alkylSO₂NH₂, —CONH₂, —CO(C₁-C₆)alkyl, —CO(C₁-C₆)haloalkyl, —CO-aryl, —CO— heteroaryl, —CO-heterocyclyl, 4- to 7-membered heterocyclic ring, 7- to 14-membered bicyclic heterocyclic ring system, bridged or spiro ring system having optionally one or more than one heteroatoms;
Each of R₆, R₇, R₈, R₉, R₁₀ and R₁₁ at each occurrence independently selected from hydrogen, halogen optionally substituted groups selected from (C₁-C₆)alkyl, (C₁-C₆)haloalkyl;
In another embodiment, the present invention provides an improved process for the preparation of compound of formula (I) as described in Scheme-3:

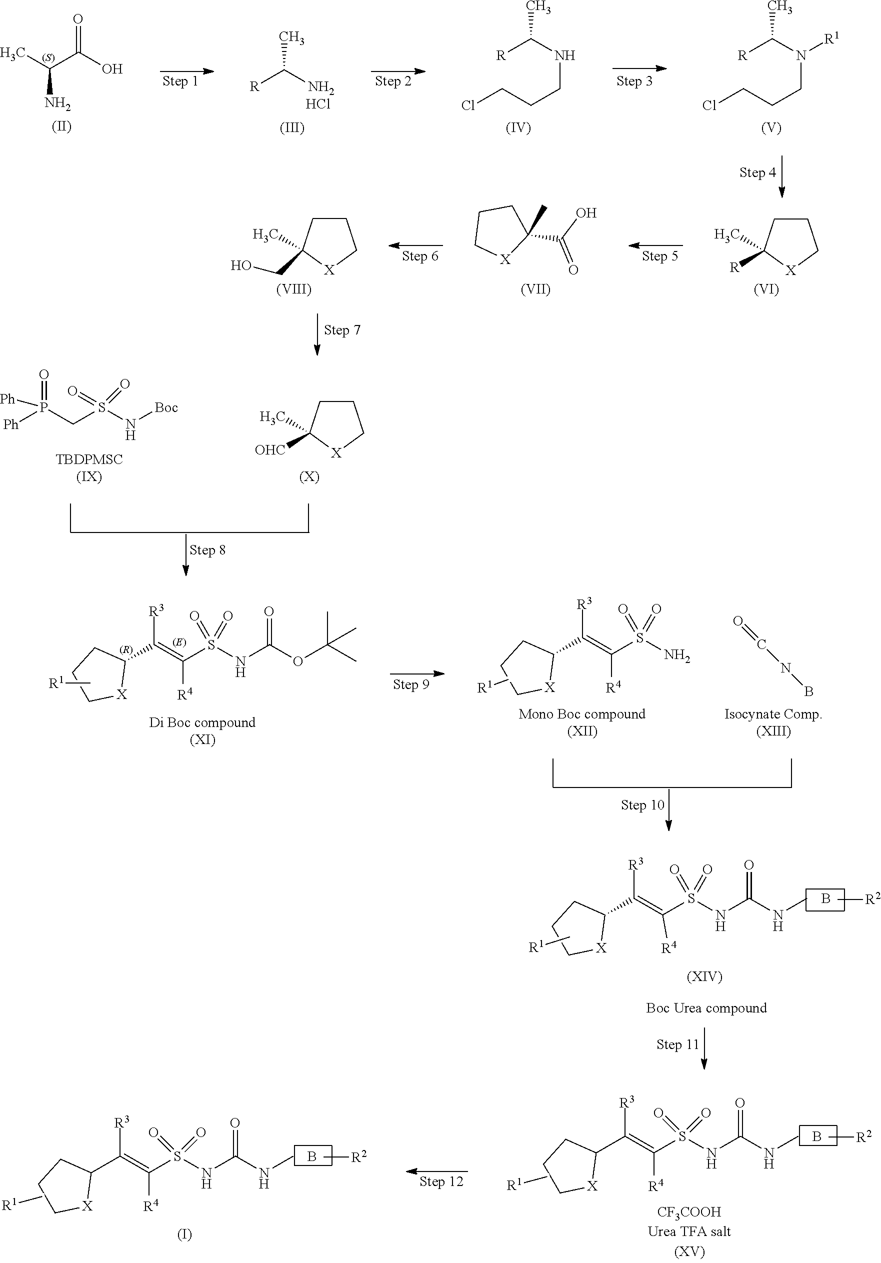

The process for the preparation of compound of formula (I) comprises the following steps:

### Step-1: Preparation of Compound (III)

Reacting compound (II) with thionyl chloride in presence of one or more solvents to obtain compound of formula (III).

In general, the organic solvent used in step-1 is selected from methanol, ethanol, isopropyl alcohol, n-butanol, ethyl acetate, toluene, tetrahydrofuran, diisopropyl ether, methyl tertiary butyl ether and the like.

### Step-2: Preparation of Compound (IV)

Reacting compound (III) with 1-bromo-3-chloropropane in presence of base and one or more solvent to obtain compound (IV).

In general, one or more solvent used in step-2 is selected from acetonitrile, toluene, ethyl acetate, dichloromethane, tetrahydrofuran, acetone, N,N-dimethylformamide, n-hexane, n-heptane and the like.

The base used in step-2 is selected from N,N-diisopropylethylamine, triethylamine, pyridine, sodium hydroxide, potassium hydroxide, sodium bicarbonate, potassium bicarbonate, DBU, DBN, DABCO and the like.

### Step-3: Preparation of Compound (V)

Reacting compound (IV) with Boc anhydride in presence of base to obtain compound of formula (V).

The base used in step-3 is selected from imidazole, N,N-diisopropylethylamine, triethylamine, pyridine, DBU, DBN, DABCO and the like.

### Step-4: Preparation of Compound (VI)

Compound (V) is dissolved in one or more solvent in presence of base to obtain compound of (VI).

One or more solvent used in step-4 is selected from dimethyl sulfoxide, dimethylformamide, toluene, tetrahydrofuran, 1,4-dioxane, dimethyl ether, Acetone, n-hexane, n-heptane and the like. The base used in step-4 is selected from sodium hydroxide, potassium hydroxide, lithium hydroxide, calcium hydroxide, sodium carbonate, potassium bicarbonate, sodium hydride, potassium hydride, potassium tert-butoxide, or sodium pentoxide, lithium diisopropylamide, potassium bis(trimethylsilyl)amide, lithium bis(trimethylsilyl)amide, sodamide and the like.

### Step-5: Preparation of Compound (VII)

Compound (VI) is dissolved in one or more solvent in the presence of suitable base to obtain compound (VII).

In general, one or more solvent used in step-5 is selected from water, methanol, ethanol, tetrahydrofuran, isopropyl alcohol, 1,4-dioxane, acetonitrile, ethyl acetate, toluene, dichloromethane, n-hexane, n-heptane and the like.

The base used in step-5 is selected from sodium hydroxide, potassium hydroxide, lithium hydroxide, potassium tertiarybutoxide, lithium diisopropylamide, potassium bis(trimethylsilyl)amide, lithium bis(trimethylsilyl)amide, sodamide and the like.

### Step-6: Preparation of Compound (VIII)

Part-A: Reacting compound (VII) with isobutyl chloroformate in presence of base and one or more solvent to obtain filterate;
Part-B: Sodium borohydride is dissolved in one or more solvent. Adding filterate obtained from Part-A to the reaction mixture to obtain compound (VIII).

In general, one or more solvent used in step-6 is selected from ethers selected from tetrahydrofuran, 1,4-dioxane, diisopropyl ether, diethyl ether, and methyl tert-butyl ether, water, ethyl acetate and the like.

The base used in step-6 is selected N-methyl morpholine, N,N-Diisopropylethyl amine, triethyl amine, pyridine, DBU, DBN, DABCO and the like.

### Step-7: Preparation of Compound (X)

Reacting compound (VIII) with oxidizing agent in presence of one or more solvent to obtain compound (X).

In general, one or more solvent used in step-7 is selected from acetonitrile, toluene, ethyl acetate, dichloromethane, tetrahydrofuran, acetone, N,N-dimethylformamide, n-hexane, n-heptane, cyclohexane and the like.

The oxidizing agents used in step-7 is selected from pyridinium chloro chromate, Pyridinium Dichromate, chromium trioxide, Collins reagent, Swern oxidation and the like.

### Step-8: Preparation of Compound (XI)

Compound (IX) is dissolved in one or more solvent in presence of base. Compound (X) is dissolved in one or more solvent and obtained solution was adding in the mixture of compound (IX) to obtain compound (XI).

In general, one or more solvent used in step-8 is selected from N,N-dimethylformamide, dimethylsulfoxide, toluene, ethers selected from tetrahydrofuran, 1,4-dioxane, diisopropyl ether, diethyl ether, methyl tert-butyl ether and the like.

The base used in step-8 is selected from sodium hydride, sodium hydroxide, potassium hydroxide, lithium hydroxide, calcium hydroxide, sodium carbonate, potassium carbonate, sodium bicarbonate, potassium bicarbonate, potassium hydride, potassium tert-butoxide or sodium pentoxide and the like.

### Step-9: Preparation of Compound (XII)

Compound (XI) is dissolved in one or more solvent and heated at suitable temperature to obtain compound (XII).

In general, one or more solvent used in step-9 is selected from dimethylformamide, dimethylsulfoxide, toluene. ethers, methanol, ethanol, isopropanol, 2-propanol, 1-butanol, and t-butyl alcohol, ethyl acetate, tetrahydrofuran, 1,4-dioxane, diisopropyl ether, diethyl ether, and methyl tert-butyl ether, n-hexane, n-heptane and the like.

Suitable temperature used in step-9 is 50° C. to 100° C.

### Step-10: Preparation of Compound (XIV)

Compound (XII) is dissolved in one or more solvent in present of base. Compound (XIII) is dissolved in one or more solvent and obtained solution was adding dropwise in the mixture of compound (XII) to obtain compound (XIV).

In general, one or more solvent used in step-10 is selected from acetonitrile, toluene, ethyl acetate, dichloromethane, tetrahydrofuran, acetone, N,N-dimethylformamide, cyclohexane, n-hexane and the like.

The base used in step-10 is selected from N-methyl morpholine, N,N-Diisopropylethylamine, triethyl amine, N,N-diisopropylethylamine, pyridine, DBU, DBN, DABCO, sodium hydride, sodium hydroxide, potassium hydroxide, lithium hydroxide, calcium hydroxide, sodium carbonate, potassium carbonate, sodium bicarbonate, potassium bicarbonate, potassium hydride, potassium tert-butoxide, or sodium pentoxide and the like.

### Step-11: Preparation of Compound (XV)

Compound (XIV) is dissolved in one or more solvent. Adding reagent in a mixture of compound (XIV) to obtain compound (XV).

In general, one or more solvent used in step-11 is selected from dichloromethane, dichloroethane, chlorobenzene, toluene, xylene, ethylbenzene, pentane, n-hexane, n-heptane, cyclohexane, tetrahydrofuran, 1,4-dioxane, diisopropyl ether, diethylether, methyl-tert-butyl ether, ethyl acetate, tetrahydrofuran, acetone, N,N-dimethylformamide, water and the like.

The reagent used in step-11 is selected from trifluoroacetic acid, hydrochloric acid, Methane sulphonic acid, Acetic acid, formic acid and the like.

### Step-12: Preparation of Compound (I)

Compound (XV) is dissolved in one or more solvent in presence of base and reagent. Adding one or more reagent to the reaction mixture of compound (XV) to obtain compound (I).

In general, one or more solvent used in step-12 is selected from water, dichloromethane, dichloroethane, chlorobenzene, toluene, xylene, ethylbenzene, pentane, n-hexane, n-heptane, cyclohexane, tetrahydrofuran, 1,4-dioxane, diisopropyl ether, diethylether or methyl tertiary butyl ether and the like.

The base, used in step-12 is selected from triethylamine, N,N-diisopropylethylamine, pyridine, DBU, DBN, DABCO and the like.

The reagent used in step-12 is selected from p-formaldehyde, sodium borohydride, Pd/C, Raney nickel, vitride, or lithium aluminium hydride and the like.

### Purification of Compound (I):

Crude compound of formula (I) obtained from step-12 is dissolved in one or more solvent to obtain pure compound (I).

In general, one or more solvent used in purification is selected from acetonitrile, toluene, ethyl acetate, dichloromethane, tetrahydrofuran, acetone, water, dimethylformamide, n-hexane, tetrahydrofuran, 1,4-dioxane, diisopropyl ether, diethyl ether, and methyl tert-butyl ether and the like.

In yet another preferred embodiment, the invention provides an improved process for the preparation of compound of formula (I-a) as described in Scheme-3:

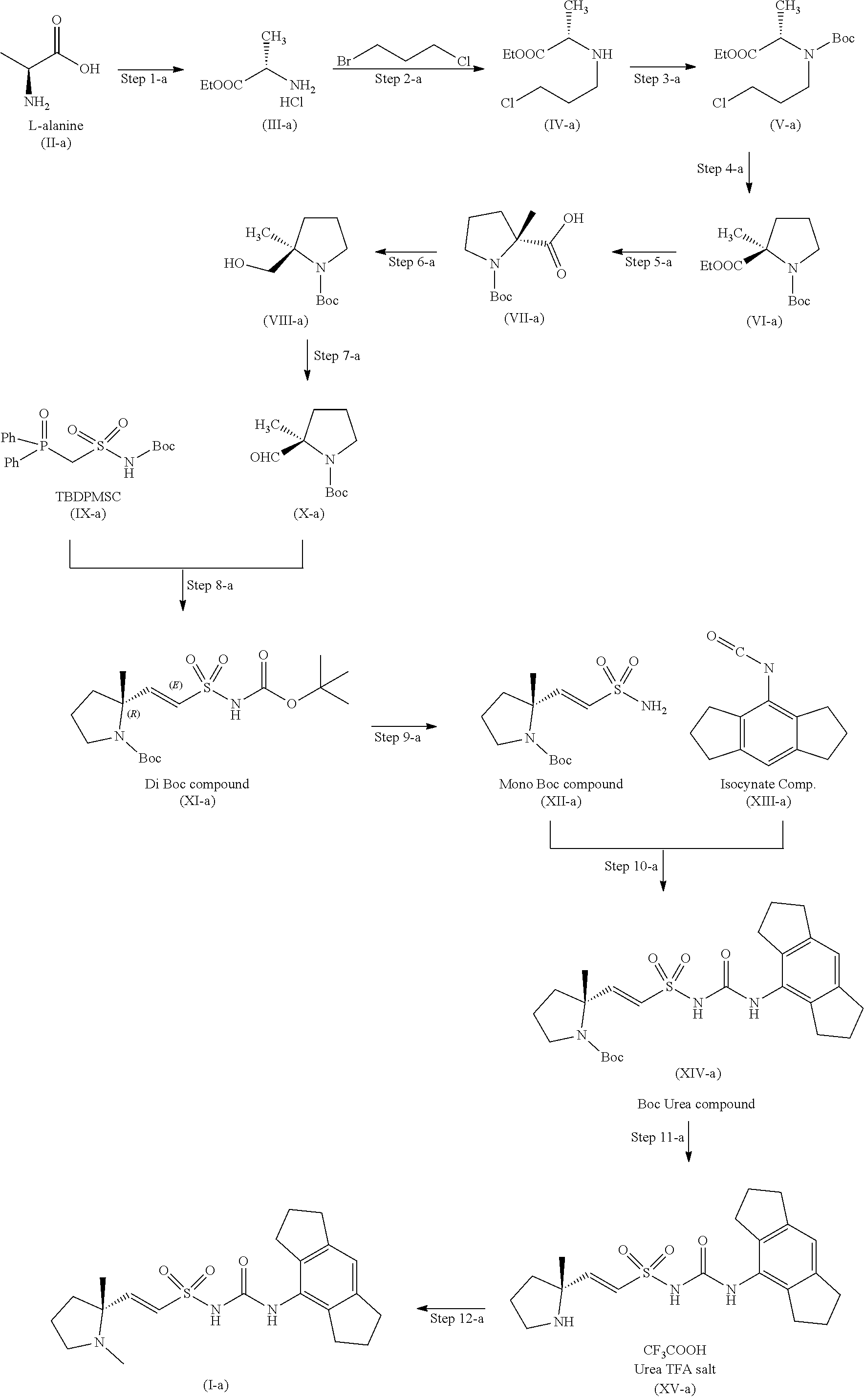

The process for the preparation of compound of formula (I-a) comprises the following steps:

### Step 1-a: Preparation of Compound (III-a)

Reacting compound (II-a) with thionyl chloride in presence of one or more solvent to obtain compound of formula (III-a);
In general, the organic solvent used in step 1-a is selected from methanol, ethanol, isopropyl alcohol, n-butanol, ethyl acetate, toluene, tetrahydrofuran, diisopropyl ether, methyl tertiary butyl ether and the like. Preferred solvent are selected from methanol, ethanol, diisopropyl ether.

### Step 2-a: Preparation of Compound (IV-a)

Reacting compound (III-a) with 1-bromo-3-chloropropane in presence of base and one or more solvent to obtain compound (IV-a).

In general, one or more solvent used in step 2-a is selected from acetonitrile, toluene, ethyl acetate, dichloromethane, tetrahydrofuran, acetone, N,N-dimethylformamide, n-hexane, n-heptane and the like. Preferred solvent are selected from acetonitrile and n-heptane.

The base used in step 2-a is selected from N,N-diisopropylethylamine, triethylamine, pyridine, sodium hydroxide, potassium hydroxide, sodium bicarbonate, potassium bicarbonate, DBU, DBN, DABCO and the like. Preferred solvent is N,N-diisopropylethylamine.

### Step 3-a: Preparation of Compound (V-a)

Reacting compound (IV-a) with Boc anhydride in presence of base to obtain compound of formula (V-a).

The base used in step 3-a is selected from imidazole, N,N-diisopropylethylamine, triethylamine, pyridine, DBU, DBN, DABCO and the like. Preferred base is Imidazole.

### Step 4-a: Preparation of Compound (VI-a)

Compound (V-a) is dissolved in one or more solvent in presence of base to obtain compound of (VI-a).

One or more solvent used in step 4-a is selected from dimethyl sulfoxide, N,N-dimethylformamide, toluene, tetrahydrofuran, 1,4-dioxane, dimethyl ether, Acetone, tetrahydrofuran, n-hexane, n-heptane and the like. Preferred solvent is dimethyl sulfoxide.

The base used in step 4-a is selected from sodium hydroxide, potassium hydroxide, lithium hydroxide, calcium hydroxide, sodium carbonate, potassium bicarbonate, sodium hydride, potassium hydride, potassium tert-butoxide, or sodium pentoxide, lithium diisopropylamide, potassium bis(trimethylsilyl)amide, lithium bis(trimethylsilyl)amide, sodamide and the like. Preferred base is potassium hydroxide.

### Step 5-a: Preparation of Compound (VII-a)

Compound (VI-a) is dissolved in one or more solvent in the presence of suitable base to obtain compound (VII-a).

In general, one or more solvent used in step 5-a is selected from water, methanol, ethanol, tetrahydrofuran, isopropyl alcohol, 1,4-dioxane, acetonitrile, ethyl acetate, toluene, dichloromethane, n-hexane, n-heptane and the like. Preferred solvent is ethanol and water.

The base used in step 5-a is selected from sodium hydroxide, potassium hydroxide, lithium hydroxide, potassium tertiarybutoxide, lithium diisopropylamide, potassium bis(trimethylsilyl)amide, lithium bis(trimethylsilyl)amide, sodamide and the like. Preferred base is potassium hydroxide.

### Step 6-a: Preparation of Compound (VIII-a)

Part-A: Reacting compound (VII-a) with isobutyl chloroformate in presence of base and one or more solvent to obtain filterate;
Part-B: Sodium borohydride is dissolved in one or more solvent. Adding filterate obtained from Part-A to the reaction mixture to obtain compound (VIII-a).

In general, one or more solvent used in step 6-a is selected from ethers selected from tetrahydrofuran, 1,4-dioxane, diisopropyl ether, diethyl ether, methyl tert-butyl ether, water, ethyl acetate and the like. Preferred solvent is tetrahydrofuran.

The base used in step 6-a is selected N-methyl morpholine, Diisopropylethyl amine, triethyl amine, potassium hydroxide, pyridine, DBU, DBN, DABCO and the like. Preferred base is N-methyl morpholine.

### Step 7-a: Preparation of Compound (X-a)

Reacting compound (VIII-a) with oxidizing agent in presence of one or more solvent to obtain compound (X-a).

In general, one or more solvent used in step 7-a is selected from acetonitrile, toluene, ethyl acetate, dichloromethane, tetrahydrofuran, acetone, N,N-dimethylformamide, n-hexane, n-heptane, cyclohexane and the like. Preferred solvent is dichloromethane.

The oxidizing agents used in step 7-a is selected from pyridinium chlorochromate, Pyridinium Dichromate, chromium trioxide, Collins reagent, Swern oxidation and the like. Preferred oxidizing agent is pyridinium chlorochromate.

### Step 8-a: Preparation of Compound (XI-a)

Compound (IX-a) is dissolved in one or more solvent in presence of base. Compound (X-a) is dissolved in one or more solvent and obtained solution was adding in the mixture of compound (IX-a) to obtain compound (XI-a).

In general, one or more solvent used in step 8-a is selected from N,N-dimethylformamide, dimethylsulfoxide, toluene, ethers selected from tetrahydrofuran, 1,4-dioxane, diisopropyl ether, diethyl ether, methyl tert-butyl ether and the like. Preferred solvent is N,N-dimethylformamide.

The base used in step 8-a is selected from sodium hydride, sodium hydroxide, potassium hydroxide, lithium hydroxide, calcium hydroxide, sodium carbonate, potassium carbonate, sodium bicarbonate, potassium bicarbonate, potassium hydride, potassium tert-butoxide or sodium pentoxide and the like. Preferred base is sodium hydride.

### Step 9-a: Preparation of Compound (XII-a):

Compound (XI-a) is dissolved in one or more solvent and heated at suitable temperature to obtain compound (XII-a).

In general, one or more solvent used in step 9-a is selected from dimethylformamide, dimethylsulfoxide, toluene. ethers, methanol, ethanol, isopropanol, 2-propanol, 1-butanol, and t-butyl alcohol, ethyl acetate, tetrahydrofuran, 1,4-dioxane, diisopropyl ether, diethyl ether, methyl tert-butyl ether, n-hexane, n-heptane and the like. Preferred solvent is dimethylsulfoxide.

Suitable temperature for heated used in step 9-a is 50° C. to 100° C.

### Step 10-a: Preparation of Compound (XIV-a)

Compound (XII-a) is dissolved in one or more solvent in present of base. Compound (XIII-a) is dissolved in one or more solvent and obtained solution was adding dropwise in the mixture of compound (XII-a) to obtain compound (XIV-a).

In general, one or more solvent used in step 10-a is selected from acetonitrile, toluene, ethyl acetate, dichloromethane, tetrahydrofuran, acetone, N,N-dimethylformamide, cyclohexane, n-hexane and the like. Preferred solvent is N,N-dimethylformamide.

The base used in step 10-a is selected from N-methyl morpholine, N,N-Diisopropylethylamine, triethyl amine, pyridine, DBU, DBN, DABCO, sodium hydride, sodium hydroxide, potassium hydroxide, lithium hydroxide, calcium hydroxide, sodium carbonate, potassium carbonate, sodium bicarbonate, potassium bicarbonate, potassium hydride, potassium tert-butoxide, or sodium pentoxide and the like. Preferred base is sodium hydride.

### Step 11-a: Preparation of Compound (XV-a)

Compound (XIV-a) is dissolved in one or more solvent. Adding reagent in a mixture of compound (XIV-a) to obtain compound (XV-a).

In general, one or more solvent used in step 11-a is selected from dichloromethane, dichloroethane, chlorobenzene, toluene, xylene, ethylbenzene, pentane, n-hexane, heptane, cyclohexane, tetrahydrofuran, 1,4-dioxane, diisopropyl ether, diethylether, methyl tert-butyl ether, ethyl acetate, tetrahydrofuran, acetone, N,N-dimethylformamide and the like. Preferred solvent is dichloromethane.

The reagent used in step 11-a is selected from trifluoroacetic acid, hydrochloric acid, Methane sulphonic acid, Acetic acid, formic acid and the like. Preferred reagent is trifluoroacetic acid.

### Step 12-a: Preparation of Compound (I-a)

Compound (XV-a) is dissolved in one or more solvent in presence of base and reagent. Adding one or more reagent to the reaction mixture of compound (XV-a) to obtain compound (I-a).

In general, one or more solvent used in step 12-a is selected from water, dichloromethane, dichloroethane, chlorobenzene, toluene, xylene, ethylbenzene, pentane, n-hexane, n-heptane, cyclohexane, tetrahydrofuran, 1, 4-dioxane, diisopropyl ether, diethylether or methyl tert-tertbutyl ether and the like. Preferred solvent is water.

The base, used in step 12-a is selected from triethylamine, Diisopropylethylamine, pyridine, DBU, DBN, DABCO and the like. Preferred base is Triethylamine.

The reagent used in step 12-a is selected from p-formaldehyde, sodium borohydride, Pd/C, Raney nickel, vitride or lithium aluminium hydride and the like. Preferred reagent is p-formaldehyde and sodium borohydride.

### Purification of Compound (I-a):

Crude compound of formula (I-a) obtained from step 12-a is dissolved in one or more solvent to obtain pure compound (I-a).

In general, one or more solvent used in purification is selected from acetonitrile, toluene, ethyl acetate, dichloromethane, tetrahydrofuran, acetone, water, dimethylformamide, n-hexane, tetrahydrofuran, 1,4-dioxane, diisopropyl ether, diethyl ether, and methyl tert-butyl ether and the like. Preferred solvent is acetone.

In one embodiment the compound of formula (I-a) either in crystalline or amorphous or partially crystalline form.

In a further embodiment of the invention disclosed a crystalline Form I of compound of formula (I-a).

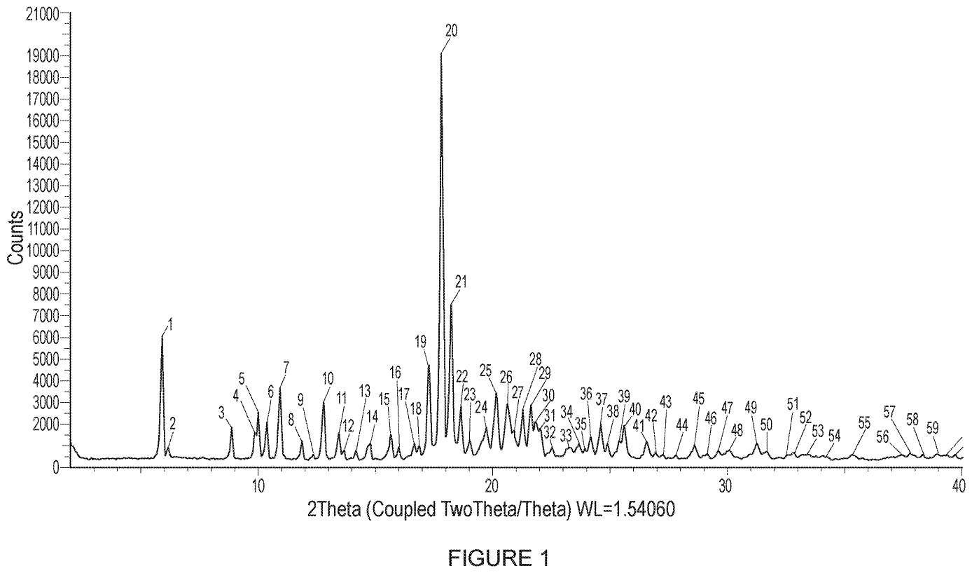

In another embodiment the crystalline Form I of formula (I-a) has a powder X-ray diffraction pattern as given in FIG. 1.

In another further embodiment the crystalline Form I of formula (I-a) has a powder X-ray diffraction pattern having a peak at about 5.9±0.2, 10.9±0.2, 17.3±0.2, 17.8±0.2, 18.2±0.2, 20.1±0.2 and 20.6±0.2 degrees 2-theta.

In yet another embodiment the crystalline Form I of formula (I-a) has a powder X-ray diffraction pattern having a peak at about 17.8±0.2 degrees 2-theta;
In yet another embodiment, the present invention provides process for the preparation of crystalline Form I of formula (I-a). The process comprises:
(i) dissolving compound of formula (I-a) in one or more solvent or a mixture of solvent to obtain crystalline Form I of formula (I-a).

solvent used in step (i) is selected from acetonitrile, toluene, ethyl acetate, dichloromethane, tetrahydrofuran, acetone, water, dimethylformamide, n-hexane, tetrahydrofuran, 1,4-dioxane, diisopropyl ether, diethyl ether, and methyl tert-butyl ether and the like.

In a further embodiment of the invention disclosed a crystalline Form II of formula (I-a).

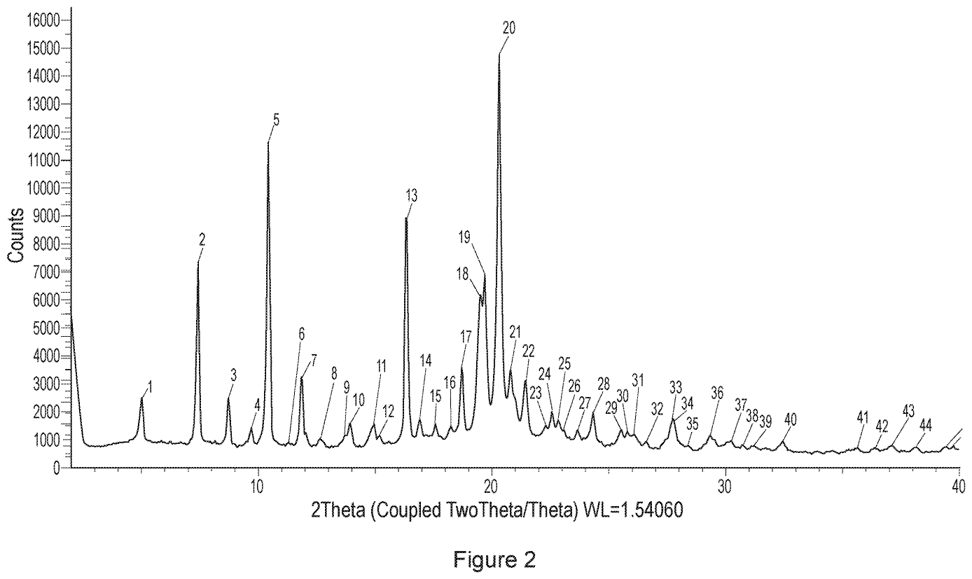

In another embodiment the crystalline Form II of formula (I-a) has a powder X-ray diffraction pattern as given in FIG. 2.

In another further embodiment the crystalline Form II of formula (I-a) has a powder X-ray diffraction pattern having a peak at about 7.4±0.2, 10.5±0.2, 16.4±0.2, 19.6±0.2, 20.4±0.2 and 20.9±0.2 degrees 2-theta.

In yet another embodiment, the present invention provides process for the preparation of crystalline Form II of formula (I-a). The process comprises:
(i) dissolving compound of formula (I-a) in one or more solvent or a mixture of solvent to obtain crystalline compound of formula (I-a).

solvent used in step (i) is selected from methanol, ethanol, Isopropyl alcohol, toluene, ethyl acetate, dichloromethane, acetone, water, and the like.

In a further embodiment of the invention disclosed a crystalline Form III of formula (I-a).

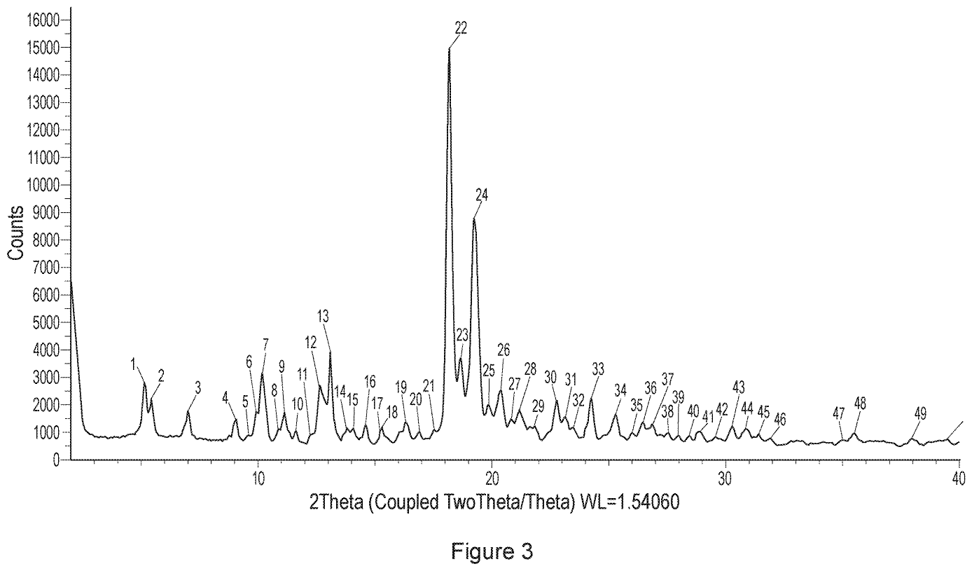

In another embodiment the crystalline Form III of formula (I-a) has a powder X-ray diffraction pattern as given in FIG. 3.

In another further embodiment the crystalline Form III of formula (I-a) has a powder X-ray diffraction pattern having a peak at about 5.2±0.2, 10.2±0.2, 13.1±0.2, 18.2±0.2, and 19.3±0.2 degrees 2-theta.

In yet another embodiment, the present invention provides process for the preparation of crystalline Form III of formula (I-a). The process comprises:
(i) dissolving compound of formula (I-a) in one or more solvent or a mixture of solvent to obtain crystalline Form III of formula (I-a).

solvent used in step (i) is selected from methanol, ethanol, isopropyl alcohol, dichloromethane, water, diisopropyl ether, diethyl ether, and methyl tert-butyl ether and the like.

In a further embodiment of the invention disclosed an amorphous form of the compound of formula (I-a).

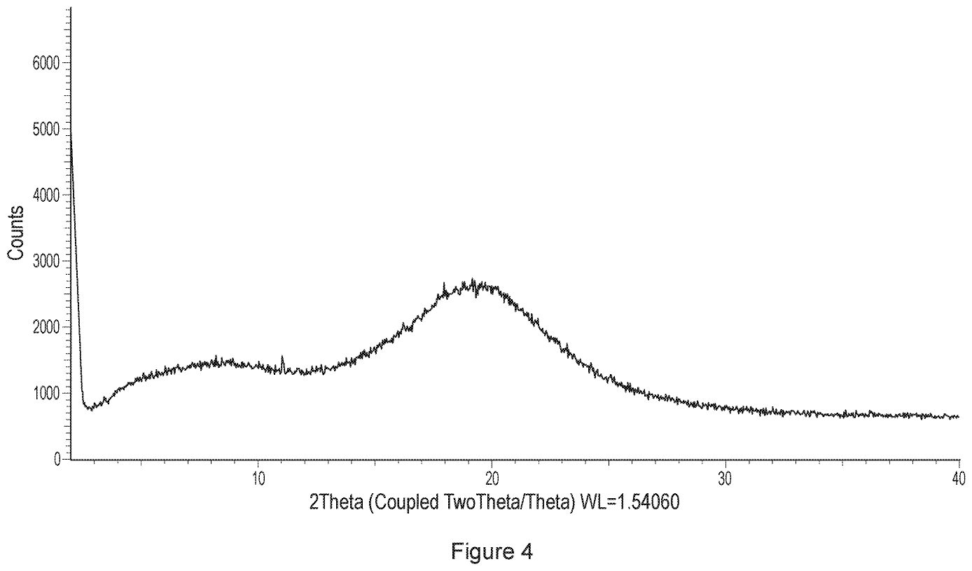

In another embodiment the amorphous form of formula (I-a) has a powder X-ray diffraction pattern as given in FIG. 4.

In yet another embodiment, the present invention provides process for the preparation of amorphous form of formula (I-a). The process comprises:
(i) dissolving compound of formula (I-a) in one or more solvent or a mixture of solvent;
(ii) spray drying of feed stock solution from step (i) to get amorphous compound of formula (I-a).

In an embodiment one or more solvent used in step (i) is selected from dichloromethane, tetrahydrofuran, acetone, water, n-hexane, tetrahydrofuran, 1,4-dioxane, diisopropyl ether, diethyl ether, methyl tert-butyl ether and the like.

In order to obtain marketing approval for a new drug product, manufacturers must submit the evidence to the regulatory authority that the product is acceptable for administration to humans. Such a submission must include impurity profile of the product to demonstrate that the impurities are either absent, or present in a negligible amount. Different regulatory authorities have promulgated guidelines requiring applicants to identify the impurities present in the product and also disclose their concentration in the product. They also provide the maximum level of impurities allowable in the product. Thus for e.g. USFDA recommends that drug applicants identify all the impurities having concentration of 0.1% or greater, in the active ingredient. Therefore, there is a need to check impurity profile and identify the impurities and also their concentration in the active ingredient.

Following novel compounds (Compound A to J) were also obtained during the synthesis of the compound of formula (I-a). These compounds may be generated as generates during process for the preparation of compound of formula (I-a).

The present invention thus provides following a new impurities of compound of formula (I-a):

(i) Compound A having chemical name (R,E)-2-(1,2-dimethylpyrrolidin-2-yl)-N-((1,2,3,7-tetrahydro-s-indacen-4-yl)carbamoyl)ethene-1-sulfonamide and having the following chemical structure:

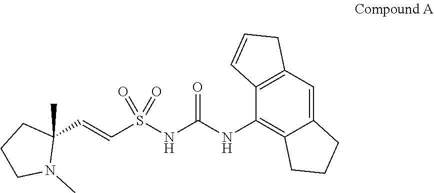 [SMILES: CN1CCC[C@]1(C)/C=C/S(=O)(=O)NC(=O)Nc1c2c(cc3c1CCC3)CC=C2]

In an embodiment the compound of formula (A) is controlled in formula (I-a) with the limit of 0.01% to 2.0%.

(ii) Compound B having chemical name (R)-2-(1,2-dimethylpyrrolidin-2-yl)-N-((1,2,3,5,6,7-hexahydro-s-indacen-4-yl)carbamoyl)ethane-1-sulfonamide and which has the following chemical structure:

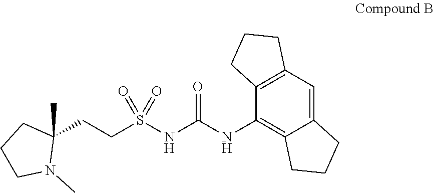 [SMILES: CN1CCC[C@]1(C)CCS(=O)(=O)NC(=O)Nc1c2c(cc3c1CCC3)CCC2]

In an embodiment the compound of formula (B) is controlled in formula (I-a) with the limit of 0.01% to 2.0%.

(iii) Compound C having chemical name 3-(1,2,3,5,6,7-hexahydro-s-indacen-4-yl)-1,1-dimethylurea and has the following chemical structure:

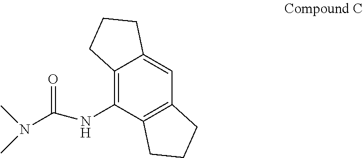 [SMILES: CN(C)C(=O)Nc1c2c(cc3c1CCC3)CCC2]

In an embodiment the compound of formula (C) is controlled in formula (I-a) with the limit of 0.01% to 2.0%.

(iv) Compound D having chemical name 1,3-bis(1,2,3,5,6,7-hexahydro-s-indacen-4-yl)urea and has the following chemical structure:

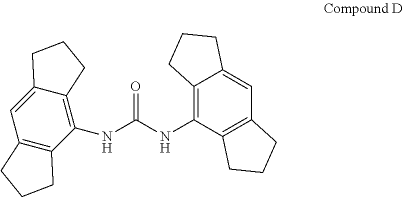 [SMILES: O=C(Nc1c2c(cc3c1CCC3)CCC2)Nc1c2c(cc3c1CCC3)CCC2]

In an embodiment the compound of formula (D) is controlled in formula (I-a) with the limit of 0.01% to 2.0%.

(v) Compound E having chemical name (S)-2-((R)-1,2-dimethylpyrrolidin-2-yl)-N-((1,2,3,5,6,7-hexahydro-s-indacen-4-yl)carbomoyl)-2-hydroxyethane-1-sulfonamide and has the following chemical structure:

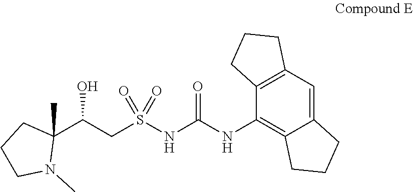 [SMILES: CN1CCC[C@]1(C)[C@H](O)CS(=O)(=O)NC(=O)Nc1c2c(cc3c1CCC3)CCC2]

In an embodiment the compound of formula (D) is controlled in formula (I-a) with the limit of 0.01% to 2.0%.

(vi) Compound F having chemical name (R)-2-((R)-1,2-dimethylpyrrolidin-2-yl)-N-((1,2,3,5,6,7-hexahydro-s-indacen-4-yl)carbamoyl)-2-hydroxyethane-1-sulfonamide and has the following chemical structure:

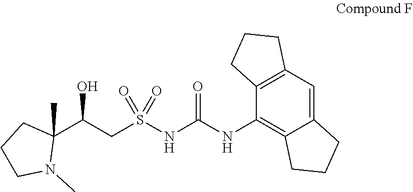 [SMILES: CN1CCC[C@]1(C)[C@@H](O)CS(=O)(=O)NC(=O)Nc1c2c(cc3c1CCC3)CCC2]

In an embodiment the compound of formula (F) is controlled in formula (I-a) with the limit of 0.01% to 2.0%.

(vii) Compound G having chemical name (R,E)-N-((1,2,3,5,6,7-hexahydro-s-indacen-4-yl)carbamoyl)-2-(2-methylpyrrolidin-2-yl)ethene-1-sulfonamide 2,2,2-trifluoroacetate and has the following chemical structure:

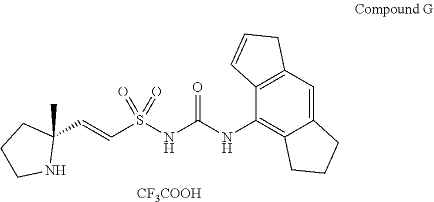 [SMILES: C[C@]1(/C=C/S(=O)(=O)NC(=O)Nc2c3c(cc4c2CCC4)CC=C3)CCCN1 ; "CF3COOH"]

In an embodiment the compound of formula (G) is controlled in formula (I-a) with the limit of 0.01% to 2.0%.

(viii) Compound H having chemical name 1,2,3,5,6,7-hexahydro-s-indacen-4-amine and has the following chemical structure:

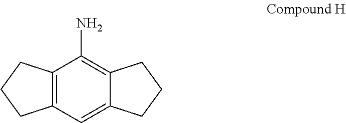 [SMILES: Nc1c2c(cc3c1CCC3)CCC2]

In an embodiment the compound of formula (H) is controlled in formula (I-a) with the limit of 0.01% to 2.0%.

(ix) Compound I having chemical name (R,E)-2-(1,2-dimethylpyrrolidin-2-yl)ethene-1-sulfonamide and has the following chemical structure:

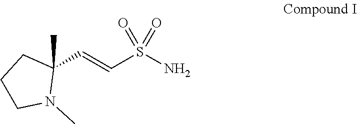

In an embodiment the compound of formula (I) is controlled in formula (I-a) with the limit of 0.01% to 2.0%.

(x) Compound J having chemical name (S,E)-2-(1,2-dimethylpyrrolidin-2-yl)-N-((1,2,3,5,6,7-hexahydro-s-indacen-4-yl)carbamoyl)ethene-1-sulfonamide and has the following chemical structure:

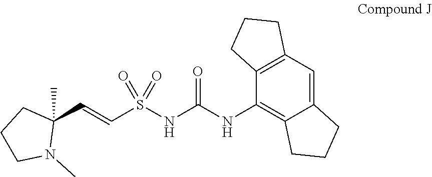 [SMILES: CN1CCC[C@@]1(C)/C=C/S(=O)(=O)NC(=O)Nc1c2c(cc3c1CCC3)CCC2]

In an embodiment the compound of formula (J) is controlled in formula (I-a) with the limit of 0.01% to 2.0%.

The invention is further exemplified by the following non-limiting examples, which are illustrative representing the preferred modes of carrying out the invention. The invention's scope is not limited to these specific embodiments only but should be read in conjunction with what is disclosed anywhere else in the specification together with those information and knowledge which are within the general understanding of a person skilled in the art.

### Example 1: Process for Preparation of Ethyl L-Alaninate Hydrochloride—Compound of Formula (III-a)

L-alanine (3.0 Kg, 33.67 mol) was added in ethanol (30 L) in a 100 L glass assembly at room temperature. Reaction mixture was cooled to 0° C. to 5° C. Thionyl chloride (12.02 Kg, 101 mol) was added carefully at 0° C. to 5° C. over a period of 45 minutes. Heated the reaction mixture to 80 to 85° C. for 3 h. Concentrated the reaction mixture in vacuum below at 65° C. to get oily residue which cool to 35 to 45° C. Diisopropyl ether (15 L×2) was added in to oily residue to get off white colored solid and decant solvent. Dry the solid in vacuum to get ethyl L-alaninate hydrochloride compound of (III-a) 5.10 Kg.

Yield: 98.6%

Appearance: Off white colored solid.

MS (ESI-MS): m/z 118.10 (M−HCl+H)⁺.

¹H NMR (400 MHz, DMSO-d₆): δ 1.22 (t, J=7.2 Hz, 3H), 1.42 (d, J=7.2 Hz, 3H), 3.98 (q, J=14 Hz, 7.2 Hz, 1H), 4.21-4.13 (m, 2H), 8.75 (s, 3H).

### Example 2: Process for Preparation of ethyl (3-chloropropyl)-L-alaninate—compound of formula (IV-a)

Ethyl L-alaninate hydrochloride (III-a) (5.1 Kg, 33.2 mol), diisopropylethyl amine (12.86 kg, 99.60 mol), 1-Bromo-3-chloropropane (18.29 kg, 116.20 mol) and tetrabutylammonium bromide (0.535 Kg, 1.660 mol) were added in acetonitrile (20.4 L) in 150 L SSR at room temperature. Reaction mixture was stirred for 72 h at room temperature and further quenched with 20% aqueous citric acid solution (127.5 L) to bring pH 3 to 4. Reaction mixture was then washed with n-Heptane (20.4 L×2) and aqueous layer was then basify with 4 N sodium hydroxide solution (95 L) to bring pH 9 to 10. Aqueous layer was extracted with methyl tertiary butyl ether (25.5 L×2). Organic layer was washed with 10% aqueous sodium bicarbonate solution (15.5 L) and 10% sodium chloride solution (10.2 L). Organic layer containing ethyl (3-chloropropyl)-L-alaninate (IV-a) was used in next reaction without any further purification.

### Example 3: Process for Preparation of ethyl N-(tert-butoxycarbonyl)-N-(3-chloropropyl)-L-alaninate—compound of formula (V-a)

Organic layer obtained from stage 2 was charged in a 150 L SSR, at room temperature. Boc anhydride (6.81 kg, 31.2 mol) was added and the reaction mixture was stirred for 16 h at room temperature. Imidazole (0.565 Kg, 8.30 mol) was added and the reaction mixture was stirred for 2 h at room temperature. The reaction mixture was washed with aqueous 1 N hydrochloric acid solution (11.2 L×3). Organic layer was washed with 5% aqueous sodium bicarbonate solution (11.25 L) followed by water (11.25 L) and 10% brine solution (11.25 L). Concentrated solvents in vacuum to get ethyl N-(tert-butoxycarbonyl)-N-(3-chloropropyl)-L-alaninate (V-a) 7.51 Kg.

Yield: 77% (two steps yield)

Appearance: Pale yellow colored oil

MS (ESI-MS): m/z 194.09 (M-Boc+H)⁺

¹H NMR (400 MHz, CDCl₃): δ 1.24 (d, J=6.8 Hz, 3H), 1.42-1.38 (m, 12H), 2.08-2.00 (m, 2H), 3.20 (d, J=6.0 Hz, 1H), 3.65-3.43 (m, 3H), 4.17-4.09 (m, 2H).

### Example 4: Process for Preparation of 1-(tert-butyl) 2-ethyl (R)-2-methylpyrrolidine-1,2-dicarboxylate—compound of formula (VI-a)

Ethyl N-(tert-butoxycarbonyl)-N-(3-chloropropyl)-L-alaninate (V-a) (5 Kg, 17.01 mol) was added in dimethyl sulfoxide (25 L) in a 50 L SSR, at room temperature. Reaction mixture was cooled at 20 to 25° C. and potassium hydroxide (2.86 Kg, 51.05 mol) was added at 20 to 25° C. and further reaction mixture was stirred for 3 h at room temperature. The reaction mixture was dump in to water (125 L) and aqueous layer was extracted with ethyl acetate (25 L×3). Organic layer was washed with 10% aqueous sodium chloride solution (25 L) and concentrated in vacuum to get 1-(tert-butyl) 2-ethyl (R)-2-methylpyrrolidine-1, 2-dicarboxylate (VI-a) 4.160 Kg.

Yield: 95%

Appearance: Oily compound

MS (ESI-MS): m/z 280.16 (M+Na)⁺

¹H NMR (CDCl₃): δ 1.29-1.22 (m, 3H), 1.43 (d, J=14 Hz, 9H), 1.54 (d, J=18.8 Hz, 3H), 1.95-1.85 (m, 3H), 2.19-2.14 (m, 1H), 3.60-3.48 (m, 2H), 4.23-4.08 (m, 2H)

### Example 5: Process for Preparation of (R)-1-(tert-butoxycarbonyl)-2-methylpyrrolidine-2-carboxylic acid—compound of formula (VII-a)

1-(tert-butyl) 2-ethyl (R)-2-methylpyrrolidine-1, 2-dicarboxylate (VI-a) (6.2 Kg, 24.093 mol) was added to the mixture of ethanol (12.4 L) and water (12.4 L) in 150 L SSR at room temperature and the reaction mixture was further cooled at 15 to 20° C. Potassium hydroxide powder (13.5 Kg, 240.93 mol) was added at 15 to 35° C. and the reaction mixture was stirred for 16 h at room temperature. The reaction mixture was quenched with water (124 L) and aqueous layer was washed with dichloromethane (31 L×2) and then aqueous layer was acidifying with 5 N aqueous hydrochloric acid (52.7 L) to bring pH 3 to 4. Aqueous layer was extracted with ethyl acetate (62 L×1 and 31 L×2). Combined all organic layer was concentrated in vacuum to get crude solid product which was recrystallized with n-heptane (25.6 L) followed by cyclohexane (24 L). Filter the solid and dry it at 50-55° C. for 16 h to get (R)-1-(tert-butoxycarbonyl)-2-methylpyrrolidine-2-carboxylic acid (VII-a) 4.255 Kg.

Yield: 77%

Appearance: Off white colored solid

MS (ESI-MS): m/z 228.11 (M−H)⁺

¹H NMR (400 MHz, MeOD): δ 1.45 (d, J=7.6 Hz, 9H), 1.53 (d, J=10.4 Hz, 3H), 2.02-1.89 (m, 3H), 2.27-2.18 (m, 1H), 3.56-3.47 (m, 2H)

HPLC Purity: 99.54%

Chiral Purity: 100.00%

SOR+5.41° (0.5% in Methanol at 20° C.)

### Example 6: Process for Preparation of tert-butyl (R)-2-(hydroxymethyl)-2-methylpyrrolidine-1-carboxylate compound of formula (VIII-a)

(R)-1-(tert-butoxycarbonyl)-2-methylpyrrolidine-2-carboxylic acid—compound of formula (VII-a) (3.75 Kg, 16.36 mol) was added in tetrahydrofuran (37.5 L) in 100 L GAR at room temperature under N₂ atmosphere the reaction mixture was cooled at 0 to 10° C. N-methyl morpholine (2.158 L, 19.63 mol.) was added followed by addition of isobutyl chloroformate (2.58 L, 19.63 mol.) at 0 to 10° C. Reaction mass was stir at 0 to 10° C. for 45 min. Reaction mixture was filtered and washed with THF (9.4 L×2) Collect filtrate i.e. Part A Sodium borohydride (1.547 Kg, 40.9 mol.) was added in water (8.8 L, 488 mol.) in 100 L GAR at room temperature. The reaction mixture was cooled to 0 to 10° C., Part-A filtrate was added dropwise at 0 to 10° C., reaction mass was stir at room temperature for 2 h. Reaction mass was quenched with water (22.5 L) filtered it, aqueous layer was extracted with ethyl acetate (37.5 L×3). Combined organic layers were washed with brine (37.5 L), aq. Layer and brine layers were re-extracted with ethyl acetate (9.36 L), Combined all organic layer was concentrated in vacuum to get tert-butyl (R)-2-(hydroxymethyl)-2-methylpyrrolidine-1-carboxylate (VIII-a) as pale yellow coloured liquid. 3.035 Kg (86% yield)

Yield: 86%

Appearance: pale yellow coloured liquid

MS (ESI-MS): m/z 228.12 (M−H)⁺

SOR (0.6% in chloroform at 21° C.): +7.68°

### Example 7: Process for Preparation of tert-butyl (R)-2-formyl-2-methylpyrrolidine-1-carboxylate compound of formula (X-a)

tert-butyl (R)-2-(hydroxymethyl)-2-methylpyrrolidine-1-carboxylate (XXI) (3.00 Kg, 13.93 mol) was dissolved in dichloromethane (30 L, 466 Vol.) in 100 L GAR at room temperature and the reaction mixture was cooled at 15 to 20° C. Pyridinium chloro chromate (6.01 Kg, 27.9 mol.) was added portionwise at 15 to 20° C. Reaction mass was stir at room temperature for 16 hrs, n-hexane (30 L) was added stirred it for 30 min and passed through celite bed, washed with n-hexane (15 L×2), Combined all organic layer was concentrated in vacuum to get tert-butyl (R)-2-formyl-2-methylpyrrolidine-1-carboxylate (V) as oily compound 2.295 Kg (77% yield).

### Example 8: Process for Preparation of tert-butyl (R,E)-2-(2-(N-(tert-butoxycarbonyl)sulfamoyl)vinyl)-2-methylpyrrolidine-1-carboxylate (XI-a)

tert-butyl (((diphenylphosphoryl)methyl)sulfonyl)carbamate (IX-a) (3.07 Kg, 7.76 mol) was added in N,N-Dimethyl formamide (23 L) in a 100 L GAR at room temperature under Nitrogen gas atmosphere, It was cooled to 0 to 5° C. and added NaH (0.988 Kg, mol). It was gradually warmed to 25° C. and stirred for 30 min. Again cooled to 0 to 5° C. and a solution of tert-butyl (R)-2-formyl-2-methylpyrrolidine-1-carboxylate (X-a) (2.3 Kg, 10.78 mol) in DMF (11.5 L) was added dropwise over a period of 0.5 h at 0 to 10° C. temp. After the addition, reaction mixture was warmed to r.t. and further stirred for 17 h. Reaction mixture was cooled to 10 to 15° C. and dumped into ice cold water (105 L) at 10 to 15° C. Aqueous layer was washed with Diisopropylether (23 L) and then aqueous layer was acidify with 50% aqueous citric acid (11 L) to bring pH 3.5 to 4.5. Aqueous layer was extracted with ethyl acetate (34.5 L×1 and 23 L×2). Organic layer was washed with water (23 L×2), 5% aqueous sodium bicarbonate solution (11.5 L×2) followed by 10% brine solution (11.5 L). Combined aqueous of sodium bicarbonate and brine wash and adjust pH 5.8 to 6.3 using aqueous citric acid. Re-extracted with ethyl acetate (5.75 L×2) Combined all organic layer was concentrated in vacuum to get tert-butyl (R,E)-2-(2-(N-(tert-butoxycarbonyl)sulfamoyl)vinyl)-2-methylpyrrolidine-1-carboxylate (XI-a) 2.680 Kg.

Yield: 63.6%

Appearance: Pale yellow colored oil

MS (ESI-MS): m/z 389.15 (M−H)⁺

### Example 9: Process for Preparation of tert-butyl (R,E)-2-methyl-2-(2-sulfamoylvinyl)pyrrolidine-1-carboxylate (XII-a)

tert-butyl(R,E)-2-(2-(N-(tert-butoxycarbonyl)sulfamoyl)vinyl)-2-methylpyrrolidine-1-carboxylate (XI-a) (2.91 Kg, 7.45 mol) was dissolved in DMSO (14.55 L, 205 mol), Water (0.145 L, 8.05 mol) & heated to 85° C. to 90° C. for 45 min. The reaction was cooled, dumped into water (72.5 L) & extracted with EtOAc (29 L×3). Combined organic layer was washed with 10% brine solution (87 L). Combined all organic layer was concentrated in vacuum and purified by Hexane/Diisopropylether to get (R,E)-2-methyl-2-(2-sulfamoylvinyl)pyrrolidine-1-carboxylate (XII-a) 1.85 Kg.

Yield: 85%

Appearance: Off white colored solid

MS (ESI-MS): m/z 289.15 (M+H)⁺

¹H NMR (400 MHz, DMSO-d₆): δ=6.98 (s, 2H), 6.61-6.49 (m, 1H), 6.25 (d, J=15.2 Hz, 1H), 3.43-3.35 (m, 2H), 1.99-1.66 (m, 4H), 1.47-1.43 (m, 3H), 1.40-1.37 (m, 9H).

### Example 10: Process for Preparation of tert-butyl (R,E)-2-(2-(N-((1,2,3,5,6,7-hexahydro-s-indacen-4-yl)carbamoyl)sulfamoyl)vinyl)-2-methylpyrrolidine-1-carboxylate (XIV-a)

To a solution of the tert-butyl (R,E)-2-methyl-2-(2-sulfamoylvinyl)pyrrolidine-lcarboxylate (XIII-a) (1.7 Kg, 5.85 mol) in DMF (13.6 bn L) at 0 to 5° C. was added NaH (60% dispersion in mineral oil) (0.307 Kg, 7.03 mol). The reaction was allowed to warm to r.t. and stirred for 30 minutes. Solution of 4-isocyanato-1,2,3,5,6,7-hexahydro-s-indacene (1.458 Kg, 7.32 mol) in N,N-dimethylformamide (1.7 L) was added dropwise at 0 to 5° C. the reaction mixture was warm to room temperature and stirred for 16 h. Reaction mixture was cooled to 0 to 5° C., cold water (6.8 L) was added at 0 to 10° C., dumped reaction mixture into water (78.2 L) at 25 to 35° C. Stirred it for 1 h at room temperature. Filtered it and washed with water (17 L×2). Combined filtrate and acidify with 50% aqueous citric acid (9.0 L) to adjust pH 3 to 4, stirred it for 1 h at room temperature. precipitate was filtered through Nutsch filter, purified with hexane & dried to get tert-butyl (R,E)-2-(2-(N-((1,2,3,5,6,7-hexahydro-s-indacen-4-yl)carbamoyl)sulfamoyl)vinyl)-2-methylpyrrolidine-1-carboxylate (XIV-a) 2.606 Kg

Yield: 91%

Appearance: Pale cream colored solid

MS (ESI-MS): m/z 488.18 (M−H)⁺

¹H NMR (400 MHz, DMSO-d₆): δ 10.41 (s, 1H), 8.06 (s, 1H), 6.96 (s, 1H), 6.87-6.77 (m, 1H), 6.55 (d, J 15.2 Hz), 3.43-3.37 (m, 2H), 2.81 (t, J 6.8 Hz, 4H), 2.67 (t, J=6.8 Hz, 4H), 2.00-1.93 (m, 5H), 1.86-1.65 (m, 3H), 1.41-1.43 (m, 3H), 1.40-1.38 (s, 9H).

### Example 11: Process for Preparation of (R,E)-N-((1,2,3,5,6,7-hexahydro-s-indacen-4-yl)carbamoyl)-2-(2-methylpyrrolidin-2-yl)ethene-1-sulfonamide 2,2,2-trifluoroacetate (XV-a)

To a solution of tert-butyl (R,E)-2-(2-(N-((1,2,3,5,6,7-hexahydro-s-indacen-4-yl)carbamoyl)sulfamoyl)vinyl)-2-methylpyrrolidine-1-carboxylate (XIV-a) (2.6 Kg, 5.31 mol) in DCM (13 L) was added trifluoroacetic acid (4.09 L, 53.1 mol) and stirred for 2 h at room temperature. Dumped reaction mixture into mixture of water (52 L) and diisopropylether (65 L) at room temperature. Stirred it for 1 h at room temperature, filtered it, washed with diisopropylether (5.2 L×2) and dried to get (R,E)-N-((1,2,3,5,6,7-hexahydro-s-indacen-4-yl)carbamoyl)-2-(2-methylpyrrolidin-2-yl)ethene-1-sulfonamide 2,2,2-trifluoroacetate (XV-a) 2.315 Kg

Yield: 87%

Appearance: Off white colored solid

MS (ESI-MS): m/z 390.13 (M+H)⁺.

¹H NMR (DMSO-d₆): δ=9.218 (bs, 2H), 8.715 (s, 1H), 7.126-6.805 (m, 2H), 3.383-3.317 (m, 1H), 3.285-3.221 (m, 1H), 2.704-2.667- (m, 4H), 2.139-1.903 (m, 8H), 1.489 (s, 3H).

### Example 12: Process for Preparation of Form I of (R,E)-2-(1,2-dimethylpyrrolidin-2-yl)-N-((1,2,3,5,6,7-hexahydro-s-indacen-4-yl)carbamoyl)ethene-1-sulfonamide (I-a)

(R,E)-N-((1,2,3,5,6,7-hexahydro-s-indacen-4-yl)carbamoyl)-2-(2-methylpyrrolidin-2-yl)ethene-1-sulfonamide compound 2,2,2-trifluoroacetaldehyde (XV-a) (2.3 kg, 4.57 mol) was added to water (23 L) in 100 L GAR at room temperature, added triethylamine (0.764 L, 5.48 mol) followed by paraformaldehyde (1.097 kg, 36.5 mol) and stir the reaction mixture was room temperature for 30 min. and the reaction mixture was further cooled at 10 to 15° C. Sodium borohydride (0.518 kg, 13.70 mol) was added at 10 to 15° C. and the reaction mixture was stirred for 2 h at room temperature. The reaction mixture was filtered and wash with water (2.3 L) and then aqueous layer was acidifying with 20% citric acid soln. (5.1 L) to bring pH 7 to 7.5. Stir the reaction mixture was 1 h at room temperature. Filter the solid and dry it at 55-60° C. for 10-12 h to get (R,E)-2-(1,2-dimethylpyrrolidin-2-yl)-N-((1,2,3,5,6,7-hexahydro-s-indacen-4-yl)carbamoyl)ethene-1-sulfonamide (I-a) (crude) 1.326 Kg, (R,E)-2-(1,2-dimethylpyrrolidin-2-yl)-N-((1,2,3,5,6,7-hexahydro-s-indacen-4-yl)carbamoyl)ethene-1-sulfonamide (I-a) (crude) 1.320 Kg was added in Acetone (13.20 L) a (10 vol.) in 20 L 4NRBF and refluxed for 2 h and further stirred for 2 h at room temperature. The reaction mixture was filtered and wash with Acetone (1.32 L) followed by Diisopropyl ether (2.64 L×2) and dry it at 55-60° C. for 12-16 h to get Form I of (R,E)-2-(1,2-dimethylpyrrolidin-2-yl)-N-((1,2,3,5,6,7-hexahydro-s-indacen-4-yl)carbamoyl)ethene-1-sulfonamide (I-a) (pure) 1.181 Kg

Yield: 64%

HPLC=98.22%

Assay by HPLC=97.5%

¹H NMR (D2O+NH3): δ=7.108 (s, 1H), 6.706-6.607 (q, 2H), 2.920-2.884 (m, 5H), 2.810-2.253 (m, 5H), 2.205, (m, 3H), 2.106-2.033 (m, 4H), 1.954-1.794 (m, 4H), 1.243 (s, 3H).

### Polymorphic Data (XRPD): Form I

| 2θ | d value | Relative Intensity |
|---|---|---|
| 5.9° ± 0.2° | 14.93 | 28.3% |
| 10.9° ± 0.2° | 8.07 | 17.3% |
| 17.3 ± 0.2° | 5.11 | 23.0% |
| 17.8 ± 0.2° | 4.95 | 100.0% |
| 18.2 ± 0.2° | 4.85 | 38.2% |
| 20.1 ± 0.2° | 4.39 | 15.4% |
| 20.6 ± 0.2° | 4.29 | 12.5% |

### Example-14: Process for preparation of Form-IT of (R,E)-2-(1,2-dimethylpyrrolidin-2-yl)-N-((1,2,3,5,6,7-hexahydro-s-indacen-4-yl)carbamoyl)ethene-1-sulfonamide (I-a)

Dissolve(R,E)-2-(1,2-dimethylpyrrolidin-2-yl)-N-((1,2,3,5,6,7-hexahydro-s-indacen-4-yl)carbamoyl)ethene-1-sulfonamide (I-a) (crude) (1.256 Kg) in a mixture of Dichloromethane (50.24 Lit.) and Methanol (1.5 Lit.) and stirrer to get clear solution and charged charcoal powder (62.8 gm) and stirred it for 30 min. at room temperature. The reaction mixture was filtered through Hyflow and wash with Dichloromethane (6.28 Lit.×2) collected filtrate and evaporated under vacuum to get solid materials. Strip out these solid materials with Acetone (1.256 Lit.×2) and degas for 1 h at 40° C. under vacuum to get 1.216 Kg of R,E)-2-(1,2-dimethylpyrrolidin-2-yl)-N-((1,2,3,5,6,7-hexahydro-s-indacen-4-yl)carbamoyl)ethene-1-sulfonamide (I-a) (Form-II)

### Polymorphic Data (XRPD): Form II

| 2θ | d value | Relative Intensity |
|---|---|---|
| 7.45° ± 0.2° | 11.84 | 45.9% |
| 10.5° ± 0.2° | 8.43 | 76.8% |
| 16.4 ± 0.2° | 5.40 | 59.6% |
| 19.6 ± 0.2° | 4.52 | 36.8% |
| 20.4 ± 0.2° | 4.35 | 100.0% |
| 20.9 ± 0.2° | 4.25 | 17.9% |

### Example-15: Process for preparation of Form-III of (R,E)-2-(1,2-dimethylpyrrolidin-2-yl)-N-((1,2,3,5,6,7-hexahydro-s-indacen-4-yl)carbamoyl)ethene-1-sulfonamide (I-a)

Charged (R,E)-2-(1,2-dimethylpyrrolidin-2-yl)-N-((1,2,3,5,6,7-hexahydro-s-indacen-4-yl)carbamoyl)ethene-1-sulfonamide (I-a) (crude) (70 g) in a mixture of Isopropyl alcohol (630 mL) and Water (70 mL) and stirred it for 1 h at room temperature. The reaction mixture was filtered and wash with Isopropyl alcohol (70 mL) followed by Diisopropyl ether (140 mL×2) and dry it at 55-60° C. for 5-6 h to get 61.5 g of (R,E)-2-(1,2-dimethylpyrrolidin-2-yl)-N-((1,2,3,5,6,7-hexahydro-s-indacen-4-yl)carbamoyl)ethene-1-sulfonamide (I-a) (Form-III)

### Polymorphic Data (XRPD): Form III

| 2θ | d value | Relative Intensity |
|---|---|---|
| 5.2° ± 0.2° | 17.3 | 14.0% |
| 10.2° ± 0.2° | 8.68 | 17.3% |
| 13.1 ± 0.2° | 6.75 | 22.8% |
| 18.2 ± 0.2° | 4.86 | 100.0% |
| 19.3 ± 0.2° | 4.59 | 56.0% |

### Example-16: Process for preparation of amorphous form of (R,E)-2-(1,2-dimethylpyrrolidin-2-yl)-N-((1,2,3,5,6,7-hexahydro-s-indacen-4-yl)carbamoyl)ethene-1-sulfonamide (I-a)

Dissolve (R,E)-N-((1,2,3,5,6,7-hexahydro-s-indacen-4-yl)carbamoyl)-2-(2-methylpyrrolidin-2-yl)ethene-1-sulfonamide (5 g) in a mixture of Tetrhydrofuran (150 mL) and Water (100 mL) to get clear solution. The resultant mass was passed through a fine filter to remove particulate matter. The solvent is removed by spray drying of feed stock solution to get amorphous (R,E)-2-(1,2-dimethylpyrrolidin-2-yl)-N-((1,2,3,5,6,7-hexahydro-s-indacen-4-yl)carbamoyl)ethene-1-sulfonamide 1.0 g.

1. A process for the preparation of a compound of formula (I): 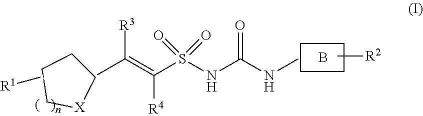
wherein
B is selected from a ring system selected from: 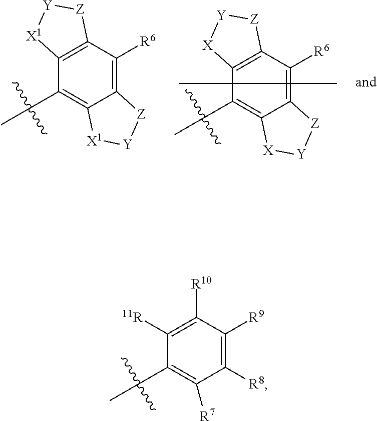
wherein each X¹, Y, and Z is independently selected from C, N, S, SO₂, and O, each of which may be optionally substituted;
each R¹ is independently selected from hydrogen, halogen, haloalkyl, and optionally substituted (C₁-C₆)alkyl;
each R² is independently selected from hydrogen, halogen, haloalkyl, and optionally substituted groups selected from (C₁-C₆)alkyl;
R³ and R⁴ are each hydrogen;
X is selected from N—R⁵; O, S, and SO₂;
each R⁵ is independently selected from hydrogen, halogen, haloalkyl, cyano, (C₁-C₆)alkyl, (C₁-C₆)haloalkyl, (C₂-C₆)alkenyl, (C₂-C₆)alkynyl, (C₁-C₆)alkoxy, (C₃-C₇)cycloalkyl, (C₁-C₆)alkylSO₂(C₁-C₆)alkyl, (C₁-C₆)alkylN(C₁-C₆)alkyl, (C₁-C₆)alkylN(C₃-C₇)cycloalkyl, aryl, heteroaryl, heterocyclyl, benzyl, thiol, mercaptoalkyl, SO₂(C₁-C₆)alkyl, SO₂(C₃-C₇)cycloalkyl, SO₂-aryl, SO₂-heterocyclyl, (C₁-C₆)thioalkyl, (C₁-C₆)thioalkoxy, (C₁-C₆)alkylSO₂NH₂, —CONH₂, —CO(C₁-C₆)alkyl, —CO(C₁-C₆)haloalkyl, —CO-aryl, —CO— heteroaryl, —CO-heterocyclyl, 4- to 7-membered heterocyclyl, 7- to 14-membered bicyclic heterocyclyl, and a bridged or spiro ring system having optionally one or more than one heteroatoms, wherein the (C₁-C₆)alkyl, (C₁-C₆)haloalkyl, (C₂-C₆)alkenyl, (C₂-C₆)alkynyl, (C₁-C₆)alkoxy, (C₃-C₇)cycloalkyl, (C₁-C₆)alkylSO₂(C₁-C₆)alkyl, (C₁-C₆)alkylN(C₁-C₆)alkyl, (C₁-C₆)alkylN(C₃-C₇)cycloalkyl, aryl, heteroaryl, heterocyclyl, benzyl, thiol, mercaptoalkyl, SO₂(C₁-C₆)alkyl, SO₂(C₃-C₇)cycloalkyl, SO₂-aryl, SO₂-heterocyclyl, (C₁-C₆)thioalkyl, (C₁-C₆)thioalkoxy, (C₁-C₆)alkylSO₂NH₂, —CONH₂, —CO(C₁-C₆)alkyl, —CO(C₁-C₆)haloalkyl, —CO-aryl, —CO-heteroaryl, —CO-heterocyclyl, 4- to 7-membered heterocyclyl, 7- to 14-membered bicyclic heterocyclyl, or the bridged or spiro ring system having optionally one or more than one heteroatoms are optionally substituted;
Each of R₆, R₇, R₈, R₉, R₁₀ and R₁₁ is independently selected from hydrogen, halogen, optionally substituted (C₁-C₆)alkyl, and optionally substituted (C₁-C₆)haloalkyl,
the process comprising the steps:
Step 1) reacting a compound of formula (II) 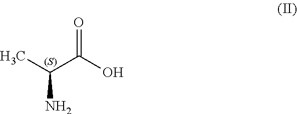
with thionyl chloride in the presence of one or more solvents to obtain a compound of formula (III); 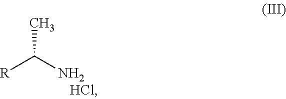
wherein R is an ester group;
Step 2) reacting the compound of formula (III) with 1-bromo-3-chloropropane in the presence of base and one or more solvents to obtain a compound of formula (IV): 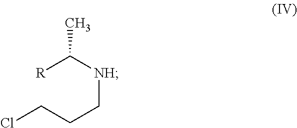
Step 3) reacting the compound of formula (IV) with Boc anhydride in presence of base to obtain a compound of formula (V) 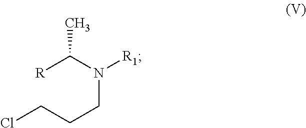
Step 4) dissolving the compound of formula (V) in one or more solvents in presence of base to obtain a compound of formula (VI): 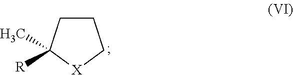
Step 5) dissolving the compound of formula (VI) in one or more solvents in the presence of a suitable base to obtain a compound of formula (VII): 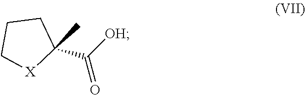
Step 6)
reacting the compound of formula (VII) with isobutyl chloroformate in the presence of base and one or more solvents to obtain a filtrate; and
adding the filtrate to a reaction mixture comprising sodium borohydride dissolved in one or more solvents to obtain a compound of formula (VIII): 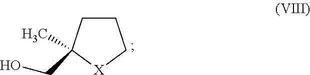
Step 7) reacting the compound of formula (VIII) with an oxidizing agent in the presence of one or more solvents to obtain a compound of formula (X): 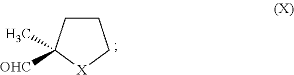
Step 8) dissolving the compound of formula (X) in a solution mixture comprising a compound of formula (IX): 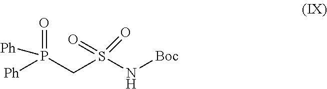 [SMILES: CC(C)(C)OC(=O)NS(=O)(=O)CP(=O)(c1ccccc1)c1ccccc1]
dissolved in one or more solvents in presence of base to obtain a compound of formula (XI): 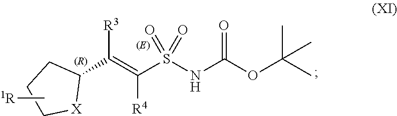
Step 9) dissolving the compound of formula (XI) in one or more solvents and heating the solution at a suitable temperature to obtain a compound of formula (XII);
Step 10) dissolving a compound of formula (XIII): 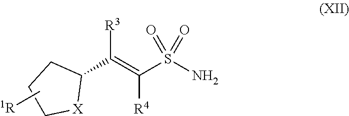
in one or solvents to form a solution comprising the compound of formula (XIII): 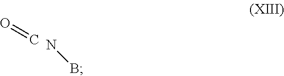
and adding the solution comprising a compound of formula (XIII) to a solution comprising the compound of formula (XII) dissolved in one or more solvents in the presence of base to obtain a compound of formula (XIV): 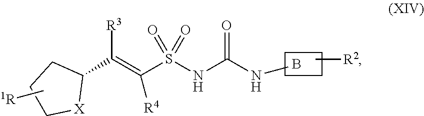
wherein the solution comprising the compound of formula (XIII) is added dropwise
Step 11) reacting the compound of formula (XIV) with a reagent in the presence of one or more solvents to obtain a compound of formula (XV): 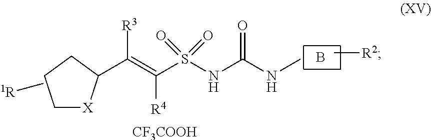
and
Step 12) reacting the compound of formula (XV) with a reagent in the presence of one or more solvents and a base to form a reaction mixture comprising a compound of formula (XV); and adding one or more reagents to the reagent mixture comprising a compound of formula (XV) to obtain a compound of formula (I).

2. The process of claim 1, wherein
(i) in Step 1, the one or more solvents is selected from methanol, ethanol, isopropyl alcohol, n-butanol, ethyl acetate, toluene, tetrahydrofuran, diisopropyl ether, methyl tertiary butyl ether;
(ii) in Step 2, the one or more solvents is selected from acetonitrile, toluene, ethyl acetate, dichloromethane, tetrahydrofuran, acetone, N,N-dimethylformamide, n-hexane, and n-heptane; and the base used is selected from N,N-diisopropylethylamine, triethylamine, pyridine, sodium hydroxide, potassium hydroxide, sodium bicarbonate, potassium bicarbonate, DBU, DBN, and DABCO;
(iii) in Step 3, the base is selected from imidazole, N,N-diisopropylethylamine, triethylamine, pyridine, DBU, DBN, and DABCO;
(iv) in Step 4, the one or more solvents is selected from dimethyl sulfoxide, dimethylformamide, toluene, tetrahydrofuran, 1,4-dioxane, dimethyl ether, acetone, n-hexane, and n-heptane; and the base used is selected from sodium hydroxide, potassium hydroxide, lithium hydroxide, calcium hydroxide, sodium carbonate, potassium bicarbonate, sodium hydride, potassium hydride, potassium tert-butoxide, or sodium pentoxide, lithium diisopropylamide, potassium bis(trimethylsilyl)amide, lithium bis(trimethylsilyl)amide, and sodamide;
(v) in Step 5, the one or more solvents is selected from water, methanol, ethanol, tetrahydrofuran, isopropyl alcohol, 1,4-dioxane, acetonitrile, ethyl acetate, toluene, dichloromethane, n-hexane, and n-heptane; and the base is selected from sodium hydroxide, potassium hydroxide, lithium hydroxide, potassium tertiarybutoxide, lithium diisopropylamide, potassium bis(trimethylsilyl)amide, lithium bis(trimethylsilyl)amide, and sodamide;
(vi) in Step 6, the one or more solvents is selected from ethers selected from tetrahydrofuran, 1,4-dioxane, diisopropyl ether, diethyl ether, and methyl tert-butyl ether, water, and ethyl acetate; and the base used is selected from N-methyl morpholine, N,N-Diisopropylethyl amine, triethyl amine, pyridine, DBU, DBN, and DABCO;
(vii) in Step 7, the one or more solvents is selected from acetonitrile, toluene, ethyl acetate, dichloromethane, tetrahydrofuran, acetone, N,N-dimethylformamide, n-hexane, n-heptane, and cyclohexane; and the oxidizing agent is selected from pyridinium chloro chromate, Pyridinium Dichromate, chromium trioxide, Collins reagent, and Swern oxidation;
(viii) in Step 8, the one or more solvents is selected from N,N-dimethylformamide, dimethylsulfoxide, toluene, ethers selected from tetrahydrofuran, 1,4-dioxane, diisopropyl ether, diethyl ether, and methyl tert-butyl ether and the base used is selected from sodium hydride, sodium hydroxide, potassium hydroxide, lithium hydroxide, calcium hydroxide, sodium carbonate, potassium carbonate, sodium bicarbonate, potassium bicarbonate, potassium hydride, potassium tert-butoxide and sodium pentoxide;
(ix) in Step 9, the one or more solvents is selected from dimethylformamide, dimethylsulfoxide, toluene, ethers, methanol, ethanol, isopropanol, 2-propanol, 1-butanol, and t-butyl alcohol, ethyl acetate, tetrahydrofuran, 1,4-dioxane, diisopropyl ether, diethyl ether, and methyl tert-butyl ether, n-hexane, and n-heptane;
(x) in Step 10, the one or more solvents is selected from acetonitrile, toluene, ethyl acetate, dichloromethane, tetrahydrofuran, acetone, N,N-dimethylformamide, cyclohexane, and n-hexane; and the base used is selected from N-methyl morpholine, N,N-Diisopropylethylamine, triethyl amine, N,N-diisopropylethylamine, pyridine, DBU, DBN, DABCO, sodium hydride, sodium hydroxide, potassium hydroxide, lithium hydroxide, calcium hydroxide, sodium carbonate, potassium carbonate, sodium bicarbonate, potassium bicarbonate, potassium hydride, potassium tert-butoxide, and sodium pentoxide;
(xi) in Step 11, the one or more solvents is selected from dichloromethane, dichloroethane, chlorobenzene, toluene, xylene, ethylbenzene, pentane, n-hexane, n-heptane, cyclohexane, tetrahydrofuran, 1,4-dioxane, diisopropyl ether, diethylether, methyl-tert-butyl ether, ethyl acetate, tetrahydrofuran, acetone, N,N-dimethylformamide, and water; and the reagent is selected from trifluoroacetic acid, hydrochloric acid, Methane sulphonic acid, acetic acid, and formic acid; or
(xii) in Step 12, the one or more solvents is selected from water, dichloromethane, dichloroethane, chlorobenzene, toluene, xylene, ethylbenzene, pentane, n-hexane, n-heptane, cyclohexane, tetrahydrofuran, 1,4-dioxane, diisopropyl ether, diethylether and methyl tertiary butyl ether; the base used is selected from triethylamine, N,N-diisopropylethylamine, pyridine, DBU, DBN, and DABCO; and the reagent is selected from p-formaldehyde, sodium borohydride, Pd/C, Raney nickel, vitride, and lithium aluminum.

3-13. (canceled)

14. The process of claim 1, wherein the process is for the preparation of a compound of formula (I-a) 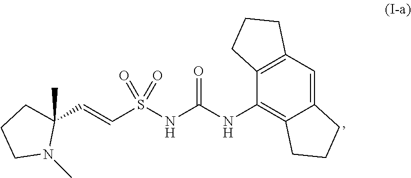 [SMILES: CN1CCC[C@]1(C)/C=C/S(=O)(=O)NC(=O)Nc1c2c(cc3c1CCC3)CCC2]
the process comprising the steps:
Step 1-a) reacting a compound of formula (II-a): 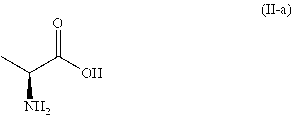
with thionyl chloride in presence of one or more solvents selected from methanol, ethanol, diisopropylether to obtain the compound of formula (III-a): 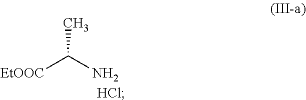
Step 2-a) reacting the compound of formula (III-a) with 1-bromo-3-chloropropane in the presence of N,N-diisopropylethylamine and one or more solvents selected from acetonitrile and n-heptane to obtain a compound of formula (IV-a): 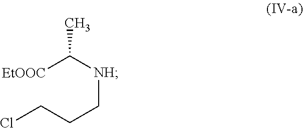
Step 3-a) reacting the compound of formula (IV-a) with Boc anhydride in presence of imidazole to obtain a compound of formula (V-a): 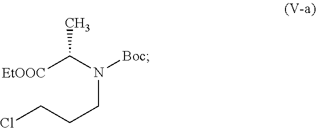 [SMILES: CCOC(=O)[C@H](C)N(CCCCl)C(=O)OC(C)(C)C]
Step 4-a) dissolving the compound of formula (V-a) in dimethylsulfoxide in the presence of potassium hydroxide to obtain a compound of formula (VI-a): 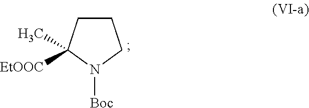 [SMILES: CCOC(=O)[C@@]1(C)CCCN1C(=O)OC(C)(C)C]
Step 5-a) dissolving the compound of formula (VI-a) in one or more solvents selected from ethanol and water in the presence of potassium hydroxide to obtain a compound of formula (VII-a): 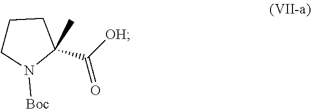 [SMILES: CC(C)(C)OC(=O)N1CCC[C@]1(C)C(=O)O]
Step 6-a)
reacting the compound of formula (VII-a) with isobutyl chloroformate in the presence of N-methyl morpholine and tetrahydrofuran to obtain a filtrate; and
adding the filtrate to a reaction mixture comprising sodium borohydride dissolved in tetrahydrofuran to obtain a compound of formula (VIII-a): 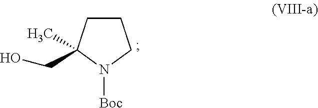
Step 7-a) reacting the compound of formula (VIII-a) with pyridinium chlorochromate in the presence of dichloromethane to obtain a compound of formula (X-a): 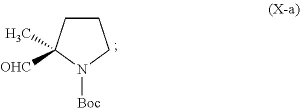
Step 8-a: dissolving the compound of formula (X-a) in a solution mixture comprising N,N-dimethylformamide and a compound of formula (IX-a): 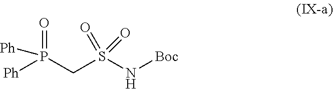 [SMILES: CC(C)(C)OC(=O)NS(=O)(=O)CP(=O)(c1ccccc1)c1ccccc1]
dissolved in N,N-dimethylformamide in the presence of sodium hydride to obtain a compound of formula (XI-a): 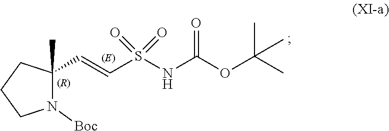
Step 9-a) dissolving a compound of formula (XI-a) dimethylsulfoxide and heating the solution at 50° C. to 100° C. to obtain a compound of formula (XII-a): 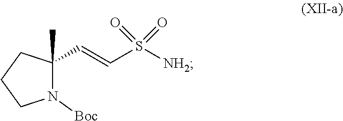 [SMILES: CC(C)(C)OC(=O)N1CCC[C@]1(C)/C=C/S(N)(=O)=O]
Step 10-a) dissolving a compound of formula (XIII-a): 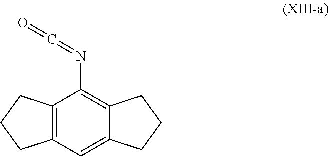 [SMILES: O=C=Nc1c2c(cc3c1CCC3)CCC2]
in N,N-dimethylformamide to form a solution comprising the compound of formula (XIII-a); and adding the solution comprising a compound of formula (XIII-a) to a solution comprising the compound of formula (XII-a) dissolved in N,N-dimethylformamide in presence of sodium hydride to obtain a compound of formula (XIV-a): 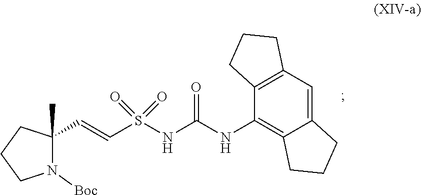 [SMILES: CC(C)(C)OC(=O)N1CCC[C@]1(C)/C=C/S(=O)(=O)NC(=O)Nc1c2c(cc3c1CCC3)CCC2]
Step 11-a) reacting the compound of formula (XIV-a) with trifluoroacetic acid in the presence of dichloromethane to obtain a compound of formula (XV-a): 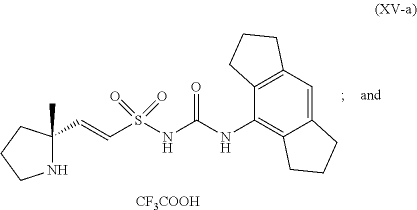 [SMILES: C[C@]1(/C=C/S(=O)(=O)NC(=O)Nc2c3c(cc4c2CCC4)CCC3)CCCN1 ; "CF3COOH"]
Step 12-a) reacting the compound of formula (XV-a) with p-formaldehyde in the presence of dissolved water and acetone in the presence of a base selected from triethylamine and diisopropylethylamine to form a reaction mixture comprising a compound of formula (XV-a); and adding sodium borohydride to the reaction mixture comprising the compound of formula (XV-a) to obtain a compound of formula (I-a).

15. A compound of formula (I-a) 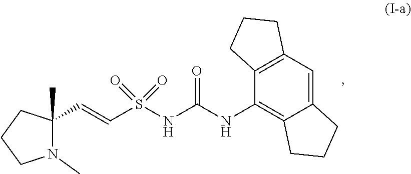 [SMILES: CN1CCC[C@]1(C)/C=C/S(=O)(=O)NC(=O)Nc1c2c(cc3c1CCC3)CCC2]
wherein the compound is in crystalline, amorphous, or partially crystalline form.

16. The compound of claim 15, wherein the compound is in crystalline Form I of formula (I-a).

17. The compound of claim 16, wherein the crystalline Form I of formula (I-a) has a powder X-ray diffraction pattern as given in FIG. 1.

18. The compound of claim 16, wherein the crystalline Form I of formula (I-a) has a characteristic powder X-ray diffraction pattern comprising peaks expressed at about 5.9±0.2, 10.9±0.2, 17.3±0.2, 17.8±0.2, 18.2±0.2, 20.1±0.2 and 20.6±0.2 degrees 2-theta.

19. The compound of claim 15, wherein the compound is in crystalline Form II of formula (I-a).

20. The compound of claim 19, wherein the crystalline Form II of formula (I-a) has a powder X-ray diffraction pattern as given in FIG. 2.

21. The compound of claim 19, wherein the crystalline Form II of formula (I-a) has a characteristic powder X-ray diffraction pattern comprising peaks expressed at about 7.4±0.2, 10.5±0.2, 16.4±0.2, 19.6±0.2, 20.4±0.2 and 20.9±0.2 degrees 2-theta.

22. The compound of claim 15, wherein the compound is in crystalline Form III of formula (I-a).

23. The compound of claim 22, wherein the crystalline Form III of formula (I-a) has a powder X-ray diffraction pattern as given in FIG. 3.

24. The compound of claim 22, wherein the crystalline Form III of formula (I-a) has a characteristic powder X-ray diffraction pattern comprising peaks expressed at about 5.1±0.2, 10.2±0.2, 13.1±0.2, 18.2±0.2, and 19.3±0.2 degrees 2-theta.

25. The compound of claim 15, wherein the compound is the amorphous compound of formula (I-a).

26. The compound of claim 25, wherein the amorphous compound of formula (I-a) has a powder X-ray diffraction pattern as given in FIG. 4.

27. A compound, wherein the compound is selected from the group consisting of:

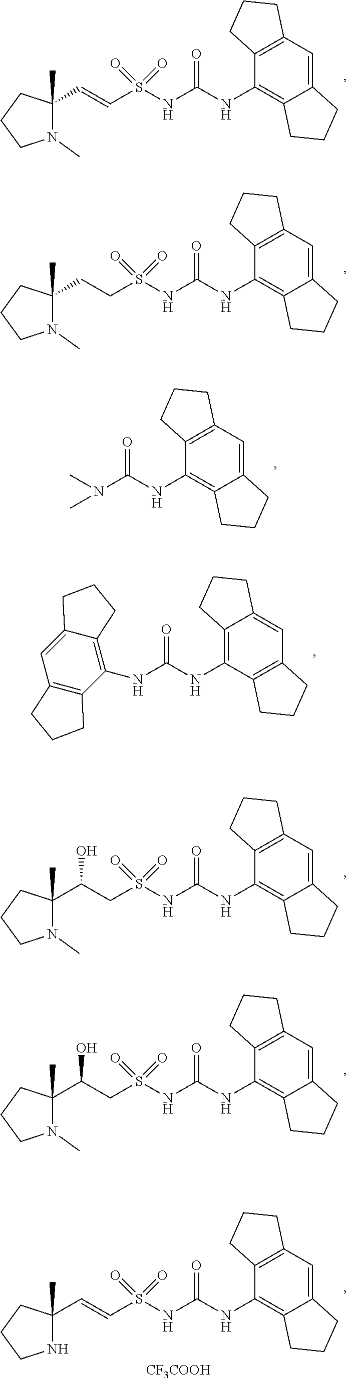

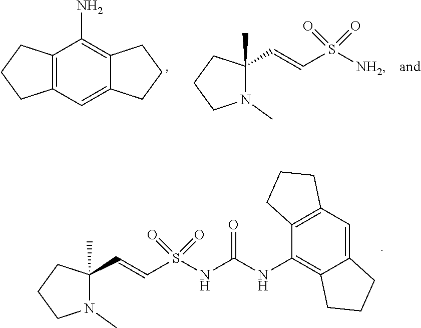

28. A composition comprising the compound of claim 15, wherein the composition further comprises a compound of claim 27, wherein the compound of claim 27 is present in an amount from 0.01 to 2.0% of the total composition.
